SEQUENCE LISTING (7 sequences)

SEQ ID NO: 1 - protein, 19 aa, Artificial Sequence
  Description of Artificial Sequence Synthetic peptide
CFTFKKEFQNNPNPRSLVK

SEQ ID NO: 2 - DNA, 36 bp, Artificial Sequence
  Description of Artificial Sequence Synthetic oligonucleotide
ggatccgctggctccgctgctggttctggcgaattc

SEQ ID NO: 3 - protein, 12 aa, Artificial Sequence
  Description of Artificial Sequence Synthetic peptide
GSAGSAAGSGEF

SEQ ID NO: 4 - protein, 16 aa, Artificial Sequence
  Description of Artificial Sequence Synthetic peptide
GGGSGGGSGGGSGGGS

SEQ ID NO: 5 - DNA, 25 bp, Artificial Sequence
  Description of Artificial Sequence Synthetic oligonucleotide
catatgtgttaactgagtaggatcc

SEQ ID NO: 6 - protein, 16 aa, Artificial Sequence
  Description of Artificial Sequence Synthetic peptide
GGGSGGGSGGGSGGGS

SEQ ID NO: 7 - protein, 8 aa, Human immunodeficiency virus type 1
LPGRWKPK

(19) United States
(12) Patent Application Publication
(10) Pub. No.: US 2006/0147997 A1
(43) Pub. Date: Jul. 6, 2006

(54) PenetraBodies: receptor-mediated targeted delivery of functionally-active human antibody fragments into cytosol for the treatment of chronic infections and diseases

(75) Inventor: Vijay Ramakrishnan, Los Altos Hills, CA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Virosys Pharmaceuticals, Inc., Los Altos Hills, CA (US)

(21) Appl. No.: 11/290,143

(22) Filed: Nov. 30, 2005

Related U.S. Application Data
(60) Provisional application No. 60/631,410, filed on Nov. 30, 2004.

(51) Int. Cl. C40B 40/10; A61K 39/02

(52) U.S. Cl. 435/7.1; 424/234.1

(57) ABSTRACT

The present invention relates to methods of making and using chimeric antibody molecules comprised of at least two domains, namely R_{AF}, and I_{AF}, linked by a flexible peptide linker.

### RELATED APPLICATION INFORMATION

This application claims the benefit of priority of Provisional Patent Application U.S. 60/631,410, filed Nov. 30, 2004. This application is hereby incorporated by reference in its entirety.

### FIELD OF THE INVENTION

Through a combination of antibody engineering and bacterial display technologies, this invention provides methods of making and using PenetraBodies for the treatment of chronic infections and diseases. PenetraBodies are specialized antibody molecules with dual specificities and have the ability to preferentially target and get internalized into specific cells, and bind to intended epitope in the cytosol.

### BACKGROUND OF THE INVENTION

Most of the biochemical processes related to chronic infections and diseases occur inside the cell, and therefore, intracellular drug targets are abound in the cytosol. Additionally, these infections and diseases occur in particular types of cells. For instance, Human Immunodeficiency Virus (HIV) specifically infects T cells expressing CD4 receptors, called CD4⁺ T cells, and macrophages (Dimitrov, Cell 101:697, 2000). Hepatitis C Virus (HCV) specifically infects liver cells and B cells (Lauer and Walker, N. Engl. J. Med. 345:41, 2001; Sung et. al., J. Virol. 77:2134, 2003).

Thus, in order to effectively target these internal epitopes in the cytosol, it is necessary that the drugs be preferentially delivered inside these cells. However, this presents several practical problems: a) first the therapeutic drug has to be targeted to a specific cell type (for example, liver cells in the case of HCV), b) the drug has to be delivered across the cell membrane and into the cytosol, and c) it has to be functional and stable under reducing conditions of the cytoplasm.

Small molecule drugs with a molecular size of ˜700 Daltons get delivered into cytosol through various non-specific uptake mechanisms. Furthermore, small molecule drugs exhibit serious side effects and toxicity because they tend to target more than one epitope in the cell. For example, the HIV drugs intended to target the HIV reverse transcriptase in cytosol also target the human DNA polymerase in mitochondria (Carr, Nature Rev. Drug Disc. 2:624, 2003). Furthermore, small molecule drugs have short serum half-life presumably in the order of minutes to hours.

In recent years, monoclonal antibodies (MAbs) have emerged as “magic bullets” to treat chronic conditions such as cancers and rheumatoid arthritis because MAbs are generally highly selective to the targets (Gura, Nature 417:584-586, 2002; Walsh, Nature Biotechnol. 21:865-870, 2003). However, because of their larger molecular size (˜150 kDa) MAbs have not been used to target the internal targets as such, and that most of the MAbs cannot be transported into cytosol. Without compromising the antigen binding property, several different MAb formats have been constructed over the years. Until recently, single chain variable fragment (scFv) was thought to be the smallest functional entity of a MAb that confers antigen binding epitope. However, recently it is proved that the variable domains of either heavy (V_{H}) or light (V_{L}) chains—the components of scFv—can itself provide antigen binding properties (Holt et. al., Trends Biotechnol. 21:484, 2003; Colby et. al., J. Mol. Biol. 342:901, 2004). The serum half-life of scFv, V_{H} or V_{L} domains is considerably shorter than that of MAbs. Through phage display technologies, internalizing scFv antibodies specific to breast tumor cell lines have been generated (Poul et. al., J. Mol. Biol. 301:1149, 2000) and have been observed to be efficiently endocytosed into SKBR3 cells, both as phage antibodies and as native monomeric scFv. Thus, the receptor-mediated endocytosis is triggered because of the specific scFvs displayed on phages.

Receptor-mediated endocytosis is a function of the protein or antibody that is expressed on bacteria and phages, and during the uptake process, the scaffolds—bacteria and phages—are also internalized. However, the efficiency of internalization differs depending on the epitope (receptor) that is recognized and the type of the antibody format used. Thus, receptor-mediated endocytosis is a promising route by which therapeutic drugs could be delivered into specific cells. Antibodies can mimic this process, stimulate endocytosis, become internalized and deliver their payload into the cytosol. In fact, scFv antibodies have been evolved through antibody engineering procedures to selectively bind and trigger endocytosis in specific cell lines (Poul et. al., J. Mol. Biol. 301:1149, 2000; Liu et. al., Cancer Res. 64:704, 2004).

Antibody fragments that target intracellular epitopes are called intrabodies. Either scFvs or the V_{H} or V_{L} domains can function as an intrabody. Given that mammalian cytosol is a reducing environment where disulfide bonds in intrabodies cannot form, only a handful of functional intrabodies have been reported to date. As a result, the vast majority of intrabodies used within a cell are not functional because they do not fold properly. One approach that has been employed in the isolation of intrabodies is the isolation of scFvs from phage display libraries followed by screening large numbers of clones for expression in E. coli or function in mammalian cells (Lecerf et. al., Proc. Natl. Acad. Sci. USA 98:4764, 2001; Gennari et. al., J. Mol. Biol. 335:193, 2004; Emadi et. al., Biochemistry 43:2871, 2004). Others have used the two-hybrid system to isolate intrabodies (Tes et. al., J. Mol. Biol. 317:85, 2002; Tanaka et. al., EMBO J. 22:1025, 2003), but this does not allow for fine-tuning of the intrabody biophysical properties such as affinity and expression.

Therefore, there remains a need for antibody molecules which can (a) preferentially target specific receptors on particular cell type(s), (b) trigger receptor-mediated endocytosis and efficiently enter into cytosol, (c) remain functionally active and specifically target the intended epitope in the cytosol, and (d) be serum stable for an extended period at physiological temperatures.

### BRIEF SUMMARY OF THE INVENTION

Provided herein are PenetraBodies, which are chimeric antibody molecules comprising at least two structurally and functionally discrete antibody domains, R_{AF} and I_{AF}, preferably connected by a flexible linker (FIG. 1). Compared to IgGs (˜150 kDa), PenetraBodies are relatively smaller in size (˜25-50 kDa) and their antigen binding properties are not compromised, thereby providing several production processing and therapeutic advantages. The present invention stems in part from the surprising discovery that, PenetraBodies are delivered to specific target cells, get internalized through receptor-mediated endocytosis, and target intracellular epitopes in the cytosol.

The R_{AF} and I_{AF} domains of the PenetraBodies may be human antibody (HuMAb) fragments comprised of any one of the following formats: scFv, V_{H}, or V_{L} domains. The R_{AF} domain binds specific receptor(s) present on particular cell types in humans, triggers receptor-mediated endocytosis, and gets internalized into cytosol. The I_{AF} domain targets an internal epitope in the cell and thus arrests the infection or progression of disease. Because I_{AF} domains are selected under reducing conditions in bacterial cytoplasm, the I_{AF} domains are active in mammalian cytosol. PenetraBodies exhibit superior pharmacokinetic and pharmacodynamic properties in addition to having extended serum half-life.

The HuMAb fragments of scFv or the V_{H} or V_{L} may be selected from bacterial display libraries. Accordingly, provided also are antibody bacterial display libraries, wherein the bacterial display, on average, has at least one copy of a scFv or V_{H} or V_{L}; the library comprises a plurality of species of scFv or V_{H} or V_{L}. Also provided are nucleic acid libraries encoding as the bacterial display antibody libraries. Further provided are processes for preparing the PenetraBodies as described above.

We have also discovered that it is possible to directly select internalizing antibodies (RIAF) from large non-immune or immune bacterial display libraries by recovering infectious internalized bacteria from within mammalian cells after receptor-mediated endocytosis (FIGS. 4, 7).

Thus, provided also are methods of selecting polypeptide or antibody binding R_{AF} domains that are internalized into specific target cells. The methods provided herein can also be used to identify internalizing receptors and/or internalizing receptor epitopes (regions of the receptor that when bound induce internalization of the binding moiety). Also provided are methods of selecting polypeptide or antibody binding moieties that target intracellular epitopes (I_{AF}).

Still further provided are methods of treating individuals with PenetraBodies, e.g. to inhibit chronic HIV and HCV infections in humans, as well as other infections and diseases. Accordingly, also provided are pharmaceutical compositions comprising the PenetraBodies and a pharmaceutically acceptable carrier.

Kits including the subject pharmaceutical compositions, and optionally instructions for their use, are also provided. Uses for such kits include, for example, therapeutic applications. In certain embodiments, the subject compositions contained in any kit have been lyophilized and require rehydration before use. Kits for the practice of the methods of preparing and selecting PenetraBodies are also provided.

Other features and advantages of the invention will be apparent from the following detailed description, and from the claims.

### BRIEF DESCRIPTION OF THE DRAWINGS

FIG. 1: Architecture of PenetraBody: PenetraBodies are composed of two functionally different antibody fragments, R_{AF} and I_{AF}, fused together by a flexible linker. To construct the R_{AF} and I_{AF} domains, scFv, or the V_{H} or V_{L} domains can be used. R_{AF}, receptor-targeting antibody fragment; I_{AF}, internal epitope-targeting antibody fragment; scFv, single chain variable fragment; V_{H}, variable fragment of heavy chain; V_{L}, variable fragment of light chain.

FIG. 2: Targeted Delivery of PenetraBody: The R_{AF} domain binds a specific receptor, triggers endocytosis, and in the process delivers I_{AF} domain to cytosol where the I_{AF} domain targets the intended epitope. The receptor and PenetraBodies can be recycled few times before they are ultimately degraded. The R_{AF} domains preferentially target a unique receptor in CD4⁺ T cells, and liver cells and B cells for treating HIV and HCV chronic infections, respectively.

FIG. 3: Structural Domains of an Immunoglobulin (IgG/MAb): MAbs are made up of a pair of heavy chains and a pair of light chains. Given that MAbs exist as discrete structural domains, they can be taken apart as “modules” but still retaining the functional specificity. One of the arms of a MAb constitutes a fragmented antibody (FAb), which has a molecular mass of ˜50 kDa. The variable regions of the heavy (V_{H}) and light (V_{L}) chains constitute the single chain variable fragment (scFv) that provides the “antigen binding property” for a MAb. Recent studies prove that either a V_{H} or V_{L} domain alone can function as the minimal entity necessary for antigen binding.

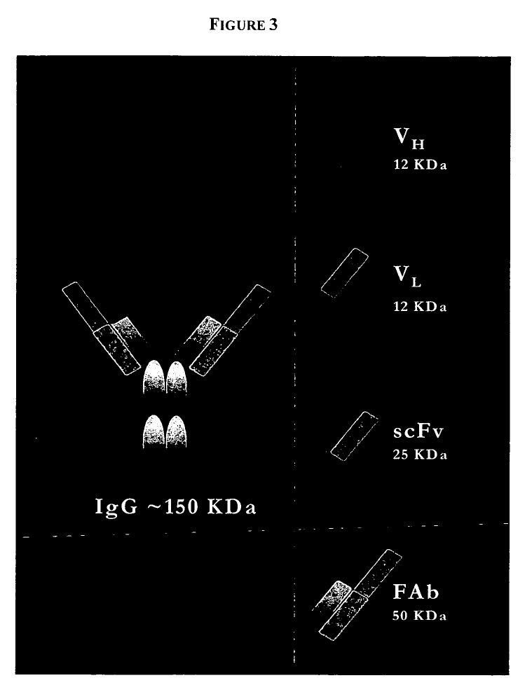

FIG. 4: Selection of R_{AF} Domain by APEx Display: HuMAb bacterial display (APEx) library is created to select R_{AF} domain, outer membrane of the E. coli removed, and E. coli cells are allowed to bind to target cells in the presence of other non-specific cells at 4° C. Receptor-mediated endocytosis is triggered by incubating the samples at physiological temperatures (37° C.). The endocytosed bacteria are isolated after extensive washing, lysing and plating of the mammalian cell lysate on bacterial plates containing appropriate antibiotics. After 4-5 such selection cycles, a high affinity R_{AF}—that not only binds to the receptor but also mediates rapid endocytosis upon binding—is selected. Alternatively, such endocytosed bacteria can also be selected by directly isolating the mammalian cells by fluorescence-activated cell sorting (FACS; Valdivia et. al., Gene 173:47, 1996). For details, see Example II.

FIG. 5: Selection of I_{AF} Domain by APEx Display: scFv is expressed as APEx bacterial display; fluorescently-labeled antigen is expressed in the periplasm of E. coli. After permeabilizing the outer membrane of E. coli, the library is subject to fluorescence-activated cell sorting (FACS). Fluorescent cells are then separated according to user-defined parameters and thus high affinity clones are selected. Only cells expressing I_{AF} species with the desired ligand-binding characteristics are enriched. See Example III.

FIG. 6: Directed Evolution of Cytoplasmically-Active I_{AF}: The I_{AF} domain selected by APEx display is then expressed in the cytoplasm of E. coli along with the reporter protein, green fluorescent protein (GFP). The fluorescence—and thus the solubility of GFP—is a function of the solubility of I_{AF}. The bacterial clones expressing fully soluble and functional I_{AF} are selected by FACS. The same approach is adopted to evolve a soluble I_{AF} from an insoluble and non-functional I_{AF} through directed evolution (see Example III).

FIG. 7: Construction of a PenetraBody Targeting HIV Reverse Transcriptase in CD4⁺ T Cells: The HIV PenetraBody described here has the R_{AF} targeting the CD4 receptor in CD4⁺ T cells and the I_{AF} targeting the reverse transcriptase of HIV inside CD4⁺ T cells. The R_{AF} is selected from an APEx display library by first subtracting the bacterial population that binds to receptors on subtractive cell lines at 4° C., and then the “depleted” library is used to pan against CD4⁺ T cells (foe example, 293 T and SupT1 cell lines). The bacteria expressing the antibodies that bind and trigger receptor-mediated endocytosis and internalization are selected, and after 4-5 reiterations, a high-affinity internalizing R_{AF} is selected. In parallel, the I_{AF} domain that targets HIV reverse transcriptase is selected by the procedures as set forth in Example III. Both R_{AF} and I_{AF} domains are fused together to form the PenetraBody (see Examples II-IV).

### DETAILED DESCRIPTION OF THE INVENTION

1. Definitions

For convenience, before further description of the present invention, certain terms employed in the specification, examples and appended claims are defined here. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to,”) unless otherwise noted.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one light (about 25 KDa) and one heavy chain (about 50-70 KDa). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively. Antibodies exist as intact immunoglobulins or as a number of well-characterized fragments produced by digestion with various peptidases. While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (scFv) in which a V_{H} and a V_{L} chains are joined together either directly or through a peptide linker to form a continuous polypeptide. The scFv antibody is a covalently linked V_{H}-V_{L} heterodimer, which may be expressed from a nucleic acid including V_{H} and V_{L}-encoding sequences either joined directly or joined by a peptide-encoding linker (Huston et. al., Proc. Nat. Acad. Sci. USA, 85: 5879, 1988). While the V_{H} and V_{L} are connected to each as a single polypeptide chain, the V_{H} and V_{L} domains associate non-covalently. The first functional antibody molecules to be expressed on the surface of filamentous phage were scFv although alternative expression strategies have been equally successful. The two chains can be encoded on the same or on different replicons.

An “antibody library” refers to bacterial display library that displays antibodies (binding proteins encoded by one or more antibody genes or cDNAs). The antibody library includes the population of bacteria or a collection of vectors encoding such a population of bacteria, or cell(s) harboring such a collection of bacteria or vectors. The library can be monovalent, displaying on average one single-chain antibody per bacterial particle or multi-valent displaying, on average, two or more single chain antibodies per bacterial particle. Preferred antibody libraries comprise on average more than 10⁶, preferably more than 10⁷, more preferably more than 10⁸, and most preferably more than 10⁹ different members (that is, encoding that many different antibodies).

An “antigen-binding site” or “paratope” refers to the part of an immunoglobulin molecule that participates in antigen binding. This paratope is formed by specific amino acid residues of the V_{H} and V_{L} chains. Alternatively, the amino acid residues from either V_{H} or V_{L} domain alone can contribute to antigen binding.

The term “APEx display” refers to anchored periplasmic expression of antibodies or protein libraries in E. coli. In APEx, proteins are expressed in the periplasm, tethered to the inner membrane of E. coli via lipidation of a small N-terminal 6-amino acid fusion.

A “bacterial display library” refers to a collection of bacteria (for example, E. coli) wherein the bacteria express an external (typically heterologous) protein. The external protein is free to interact with (bind to) other moieties with which the bacteria are contacted. Each bacterium displaying an external protein is a member of the bacterial display library.

The term “binding polypeptide” refers to a polypeptide that specifically binds to a target molecule (for example, a cell receptor) in a manner analogous to the binding of an antibody to an antigen. Binding polypeptides are distinguished from antibodies in that they are not ultimately derived from immunoglobulin genes or fragments of immunoglobulin genes.

A “chimeric molecule” is a molecule in which two or more molecules that exist separately in their native state are joined together to form a single molecule having the desired functionality of all of its constituent molecules. While the chimeric molecule may be prepared by covalently linking two molecules each synthesized separately, one of skill in the art will appreciate that where the chimeric molecule is a fusion protein, the chimera may be prepared de novo as a single “joined” molecule.

The term “conservative substitution” is used in reference to proteins or peptides to reflect amino acid substitutions that do not substantially alter the activity (specificity or binding affinity) of the molecule. Typically conservative amino acid substitutions involve substitution of one amino acid for another amino acid with similar chemical properties (e.g. charge or hydrophobicity). The following six groups each contain amino acids that are typical conservative substitutions for one another: [1] Alanine (A), Serine (S), Threonine (T); [2] Aspartic acid (D), Glutamic acid (E); [3] Asparagine (N), Glutamine (Q); [4] Arginine (R), Lysine (K); [5] Isoleucine (I), Leucine (L), Methionine (M), Valine (V); and [6] Phenylalanine (F), Tyrosine (Y), Tryptophan (W).

The term “detectable label” refers to any material having a detectable physical or chemical property. Such detectable labels have been well established in the field of immunoassays. Thus, a label is any composition detectable by spectroscopic, photochemical, biochemical, immunochemical, electrical, optical or chemical means. Useful labels in the present invention include magnetic beads, fluorescent dyes (example, fluorescein isothiocyanate, rhodamine), enzymes (example, LacZ, horse radish peroxidase, alkaline phosphatase). Those detectable labels that can be expressed by nucleic acids are referred to as ‘reporter genes’ or ‘reporter gene products’. Fluorescent labels are not to be limited to single species organic molecules, but include inorganic molecules, multi-molecular mixtures of organic and/or inorganic molecules, and the like. Thus, for example, CdSe—CdS core-shell nanocrystals enclosed in a silica shell can be easily derivatized for coupling to a biological molecule (Bruchez et. al., Science 281: 2013, 1998). Similarly, highly fluorescent quantum dots (zinc sulfide-capped cadmium selenide) have been covalently coupled to biomolecules for use in ultrasensitive biological detection (Warren and Nie, Science 281:2016, 1998; Wu et. al., Nature Biotechnol. 21:41, 2003; Jaiswal et. al., Nature Biotechnol. 21:47, 2003).

An “effector moiety” is a molecule or moiety that typically has a characteristic activity that is desired to be delivered to a target cell (e.g. CD4⁺ T cell expressing CD4 receptor). The effector molecules include antibodies, cytotoxins, drugs, and viral coat proteins.

By “effective amount” is meant the amount of PenetraBody of the invention that is useful for treating, partially or completely inhibiting, or preventing an infection in a patient or subject as described herein. Effective dosages and schedules for administering the antibodies of the invention may be determined empirically, and making such determinations is routine to one of ordinary skill in the art. An effective dose of PenetraBody generally will range between about 1 μg/kg of body weight and 25 mg/kg of body weight.

A “fusion protein” is a chimeric molecule in which the constituent molecules are all polypeptides and are attached (fused) to each other through terminal peptide bonds so that the chimeric molecule is a continuous single-chain polypeptide. The various constituents can be directly attached to each other or can be coupled through one or more peptide linkers.

By “half-life” is meant that the amount of time it takes for one-half of the drug dose to be lost through biological processes.

The term “heterologous nucleic acid” refers to a nucleic acid that is not native to the cell in which it is found.

Three highly divergent stretches within the V regions of the heavy and light chains are referred to as “hypervariable regions” which alternate between more conserved flanking stretches known as framework regions. The hypervariable regions of V_{H} and V_{L} are positioned relative to each other in three-dimensional space to form an antigen-binding pocket. This surface mediates recognition and binding of the target antigen. The three hypervariable regions of each of the V_{H} and V_{L} chains are referred to as complementarity determining regions (CDRs).

The term “I_{AF}” refers to the intrabody antibody fragment. As in R_{AF}, scFv, or the V_{H} or V_{L} domain can constitute the I_{AF}.

As used herein, the term “immunological binding” refers to the non-covalent interactions of the type which occur between an immunoglobulin molecule and an antigen for which the immunoglobulin is specific. The strength or affinity of immunological binding interactions can be expressed in terms of the dissociation constant (K_{D}) of the interaction, wherein a smaller K_{D} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the on (kₒₙ) and the off (k_{off}) rate constants can be determined by calculation of the concentrations and the actual rates of association and dissociation. The ratio of k_{off}/kₒₙ enables cancellation of all parameters not related to affinity and is thus equal to the dissociation constant K_{D} (Davies et. al., Ann. Rev. Biochem., 59: 439, 1990).

“Inhibiting HCV infection” refers to the inhibition in the onset of HCV infection, the inhibition of an increase in an existing viral infection, or a reduction in the severity of the infection. In this regard, one of ordinary skill in the art will appreciate that, while complete inhibition of the onset of HCV infection is desirable, any degree of inhibition of an increase in an existing HCV infection or any degree of a reduction of HCV infection is beneficial. Inhibition of HCV infection can be assayed by methods that are known in the art, such as by the assessment of viral load. HCV loads can be measured by methods that are known in the art, for example, using polymerase chain reaction assays to detect the presence of HCV RNA, or antibody assays to detect the presence of HCV protein in a sample (e.g. blood) from HCV-infected humans.

“Inhibiting HIV infection” refers to the inhibition in the onset of HIV infection, the inhibition of an increase in an existing viral infection, or a reduction in the severity of the infection. In this regard, one of ordinary skill in the art will appreciate that, while complete inhibition of the onset of HIV infection is desirable, any degree of inhibition of an increase in an existing HIV infection or any degree of a reduction of HIV infection is beneficial. Inhibition of HIV infection can be assayed by methods that are known in the art, such as by the assessment of viral load. HIV loads can be measured by methods that are known in the art, for example, using polymerase chain reaction assays to detect the presence of HIV RNA, or antibody assays to detect the presence of HIV protein in a sample (e.g. blood) from HIV-infected humans.

The terms “internalizing” or “internalized” when used in reference to a cell refer to the transport of a moiety (for example, bacteria) from outside to inside a cell. The internalized moiety can be located in an intracellular compartment, e.g. a vacuole, a lysosome, the endoplasmic reticulum, the golgi apparatus, or in the cytosol of the cell itself.

An “internalizing receptor” or marker is a molecule present on the external cell surface that when specifically bound by an antibody or binding protein results in the internalization of that antibody or binding protein into the cell. Internalizing receptors or markers include receptors (e.g., hormone, cytokine or growth factor receptors), ligands and other cell surface markers binding to which results in internalization. For instance, CD4 is an internalizing receptor in CD4⁺ T cells.

By “isolated polypeptide” is meant a polypeptide (or a fragment thereof) that has been separated from the components that naturally accompany it. Typically, the polypeptide is substantially pure when it is at least 60%, by weight, free from the proteins and naturally occurring organic molecules with which it is naturally associated. A substantially pure polypeptide may be obtained, for example, by extraction from natural source by expression of a recombinant nucleic acid encoding the polypeptide, or by chemically synthesizing the polypeptide. Purity can be measured by appropriate methods such as column chromatography, polyacrylamide gel electrophoresis, or HPLC analyses.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

A “non-conservative substitution” is a substitution in which the substituting amino acid (naturally or non-naturally occurring) has significantly different size, configuration and/or electronic properties compared to the amino acid being substituted. Thus, the side chain of the substituting amino acid can be significantly lower (or smaller) than the side chain of the native amino acid being substituted and/or can have functional groups with significantly different electronic properties than the amino acid being substituted.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et. al., Nucleic Acid Res. 19:5081, 1991; Ohtsuka et. al., J. Biol. Chem. 260:2605, 1985). The term nucleic acid is used interchangeably with gene, cDNA, and the mRNA encoded by a gene.

The term “PenetraBody” refers to a chimeric antibody molecule that is comprised of two domains, R_{AF} and I_{AF}, linked by a hydrophilic, peptide linker. The R_{AF} and I_{AF} domains have two distinct functions.

By “pharmacokinetics” is meant that the movement of drugs throughout the body, including their absorption, distribution, metabolism and excretion, and the mathematical models that describe these actions.

By “pharmacodynamics” is meant that changes in measurable clinical parameters related to a drug, such as decrease in viral load.

A “patient” or “subject” or “host” refers to either a human or non-human animal.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

A “pharmaceutically-acceptable salt” refers to the relatively non-toxic, inorganic and organic acid addition salts of compounds.

The terms “polypeptide”, “peptide”, or “protein” are used interchangeably herein to designate a linear sequence of amino acid residues by peptide bonds between the alpha amino and carboxy groups of adjacent residues. The amino acid residues are preferably in the natural L isomeric form. However, residues in the D isomeric form can be substituted for any L-amino acid residue, as long as the desired functional property is retained by the polypeptide.

The term “R_{AF}” refers to the receptor-targeting antibody fragment that mediates endocytosis upon binding. scFv, or a V_{H} or V_{L} domain can constitute the R_{AF}.

The term “receptor-mediated endocytosis” refers to a biological mechanism by which a ligand, upon binding to its receptor, triggers membrane invagination and pinching, gets internalized and delivered into cytosol or transferred to appropriate intracellular compartment. The ligand can be a protein, or peptide, or antibody, or membrane anchored proteins on viruses and bacteria. For example, HIV gp120 binding to CD4 receptor in T cells, when further fusion events are arrested, leads to receptor-mediated endocytosis (Schaeffer et. al., J. Virol. 78:1375, 2004).

The term “in vitro scanning saturation mutagenesis (SSM)” refers to a method particularly valuable for antibody engineering studies as a rapid way of identifying mutants with interesting properties that can be produced in large quantity and subjected to more detailed structural and functional characterization. SSM represents as a systematic new tool for exploring in vitro antibody affinity evolution, analogous to somatic hypermutation in vivo. An amino acid residue of interest in a protein sequence is mutated to twenty other possible substitutions, and its effect on the structure and function of the protein analyzed. Interesting single mutants can be used as a starting point for subsequent rounds of SSM at other sites, so that multiple mutations with synergistic effects on binding may be identified. This same sequential mutation approach should be useful to optimize properties such as affinity, potency, efficacy, altered specificity, reduced immunogenicity, and removal of proteolytic cleavage sites (U.S. Pat. No. 6,180,341).

The term “sequential (alternate) antigen panning” refers to a method by the affinity is increased step-wise for an antigen-antibody reaction without compromising the ability to bind an array of variants (Zhang et. al., J. Mol. Biol. 335:209, 2004). This method is particularly useful in HIV and HCV drug discovery programs as these viruses present themselves as distinct clinical isolates with extensive variations and mutations in protein sequences.

By “shelf-life” is meant that the amount of time a stored drug retains its activity.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

The term “specifically binds to a protein” refers to a binding reaction which is determinative of the presence of the protein in the presence of a heterogeneous population of proteins. Thus, under designated immunoassay conditions, the specified antibodies bind to a particular protein and do not bind in a significant amount to other proteins present in the sample. Specific binding to a protein under such conditions may require an antibody that is selected for its specificity for a particular protein (Harlow and Lane (1988), Antibodies: A Laboratory Manual, Cold Spring Harbor Publications, New York, N.Y.).

A protein is “substantially free” of naturally associated components when it is separated from those contaminants, which accompany it in its natural state. Thus, a protein that is chemically synthesized or produced in a cellular system different from the cell from which it naturally originates will be substantially free from its naturally associated components. Accordingly, substantially pure polypeptides not only include those derived from eukaryotic organisms but also those produced in E. coli or other prokaryotes.

A “target cell” refers to a cell or cell-type that is to be specifically bound by a member of a bacterial display library of this invention. Preferred target cells are cells for which an internalizing antibody or binding polypeptide is sought. The target cell is typically characterized by the expression or over expression of a target molecule that is characteristic of the cell type. Thus, for example, the T cell expressing CD4 receptor is a target cell.

A “targeting moiety” refers to a moiety (e.g. a molecule) that specifically binds to the target molecule. Where the target molecule is a molecule on the surface of a cell and the targeting moiety is a component of a chimeric molecule, the targeting moiety specifically binds the chimeric molecule to the cell bearing the target. Where the targeting moiety is a polypeptide it can be referred to as a targeting polypeptide. Thus, the domain R_{AF} of PenetraBody is a targeting moiety.

The following abbreviations are used herein: R_{AF}, receptor targeting antibody fragment; I_{AF}, intrabody antibody fragment; scFv, single chain variable fragment; V_{H}, variable fragment of heavy chain; V_{L}, variable fragment of light chain; APEx, anchored periplasmic expression; dAB, domain antibody; MAb, monoclonal antibody; HuMAb, human MAb; IgG, immunoglobulin; HIV, human immunodeficiency virus; HCV, hepatitis C virus; CDR, complementarity determining region; ELISA, enzyme linked immunosorbent assay; FC, flow cytometry; FACS, fluorescence activated cell sorting; SPR, surface plasmon resonance; RU, response units; FR, framework region; kₒₙ, association rate constant; k_{off}, dissociation rate constant; PCR, polymerase chain reaction; Vₖ, immunoglobulin kappa light chain variable region; V_{□}, immunoglobulin lambda light chain variable region; cfu, colony forming unit.

2. PenetraBodies and Methods of Selecting the Same

Provided are chimeric antibody molecules called PenetraBodies and compositions comprising such PenetraBodies. PenetraBodies comprise at least two structurally and functionally discrete antibody domains, R_{AF} and I_{AF}, preferably connected by a flexible linker. PenetraBodies may be constructed from human antibody (HuMAb) fragments of scFv, V_{H} or V_{L} domains, selected from bacterial display libraries. The R_{AF} domain selectively targets a specific receptor on a particular cell, gets internalized through receptor-mediated endocytosis, and released into cytosol. The I_{AF} domain is the intrabody component of the PenetraBody and binds to the intended epitope in cytosol. The R_{AF} and I_{AF} domains are linked by a flexible linker, which could also function as an endosomal escape domain. PenetraBodies may selected through a combination of protein display technologies, particularly bacterial display technology—involving the construction of anchored periplasmic expression (APEx) libraries in the bacterial periplasm as well as libraries expressed in bacterial cytoplasm—such that both R_{AF} and I_{AF} domains are properly folded and functionally active under physiologically reducing environments of the cytosol. Furthermore, upon modifying the surface-destabilizing residues of the R_{AF} and I_{AF} domains, PenetraBodies have a high degree of serum stability.

In one embodiment, the PenetraBodies are cytoplasmically-active under reducing conditions, and have an exceptional serum stability. In another embodiment, both R_{AF} and I_{AF} domains of PenetraBodies are selected through a combination of bacterial display technologies and antibody engineering procedures.

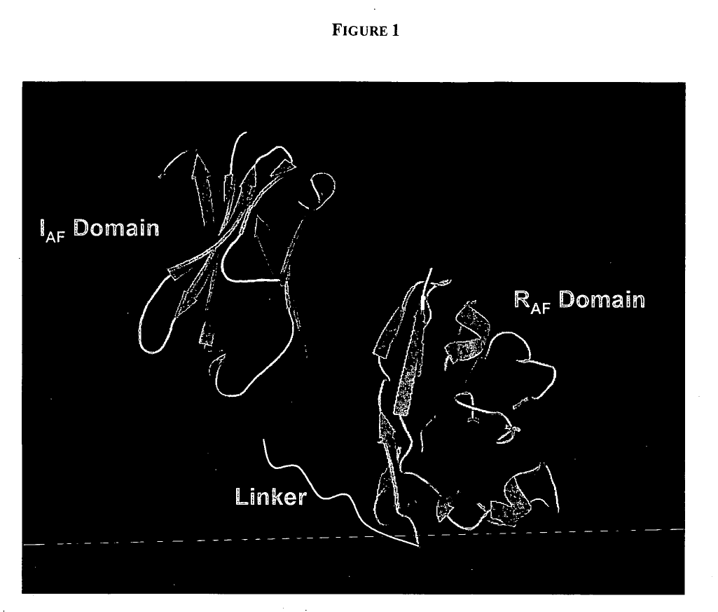

Accordingly, also provided are methods of selecting PenetraBodies. One method for making a substantially homogenous preparation of PenetraBodies comprises: (a) selection of R_{AF} domain; (b) selection of I_{AF} domain; and (c) linking R_{AF} and I_{AF} domains through a flexible linker or endosomal escape domain. Other embodiments of the methods may comprise (a) making an antibody derivative, R_{AF}, that selectively binds a specific receptor in a cell, triggers receptor-mediated endocytosis and gets internalized into cytosol, (b) making another antibody derivative, I_{AF}, that is cytoplasmically active and selectively binds to an intended epitope in cytosol, and (c) fusing the R_{AF} and I_{AF} domains with a flexible linker (FIG. 1). In developing PenetraBodies, it is necessary to select for antibodies that trigger a biological function—not just the antibodies that bind to receptors with high affinity. In this case the biological function is receptor-mediated endocytosis. Such antibodies are likely to have significant therapeutic utility.

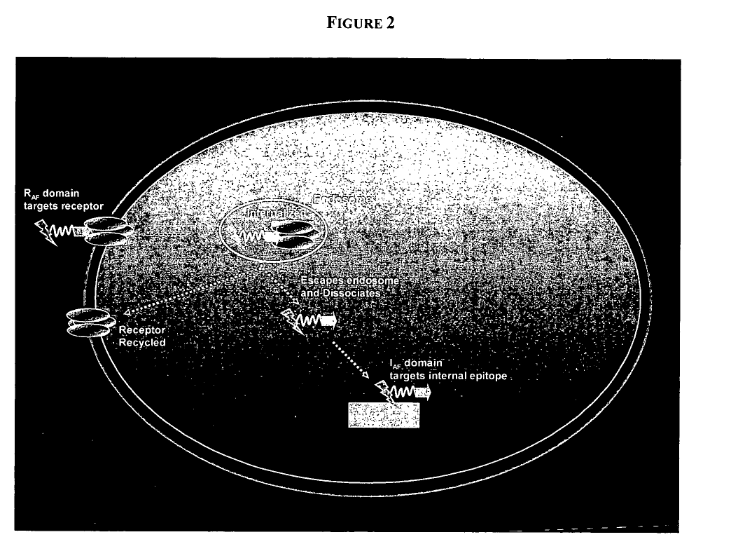

Receptor-mediated endocytosis is a highly regulated and coordinated biological event, and is triggered when a specific protein (or antibody) binds to a specific protein that acts as a receptor on a cell, and thus it is an event mediated by a protein-protein interaction (FIG. 2). In fact, when the HIV fusion process is arrested, it undergoes receptor-mediated endocytosis and is degraded in lysosomes (Schaeffer et. al., J. Virol. 78:1375, 2004). The HIV protein gp120 binds to CD4 receptor in CD4⁺ T cell to initiate this event (Schaeffer, et. al., supra). There are ˜60,000 CD4 receptors per CD4⁺ T cell and ˜2.5×10¹¹ CD4⁺ T cells in the body (Clark et. al., Adv. Immunol. 73:301, 1999; Lee et. al., Proc. Natl. Acad. Sci. USA 96:5215, 1999). As part of strategies to target HIV-infected cells, it has been previously shown that rhabdoviruses and retroviral vectors engineered to express human CD4 and CXCR4 receptors on their envelopes were able to superinfect HIV-infected cell lines, presumably by interacting with viral envelope glycoproteins on the cell surface (Schnell et. al., Cell 90:849, 1997; Endres et. al., Science 278:1462, 1997; Nolan et. al., Cell 90:821, 1997). Thus it is possible to target specific cells such as CD4⁺ T cells.

Several lines of evidence suggest that when a receptor-specific scFv or a protein is expressed on phages or E. coli, endocytosis indeed occurs, and in the process, the phage or E. coli is also endocytosed into cytosol (Ortega-Barria et. al., Cell 67:411, 1991; Ivanenkov et. al., Biochim. Biophys. Acta. 1448:463, 1999; Poul et. al, J. Mol. Biol. 301:1149, 2000). When an eukaryotic protein, penetrin, of Trypanosoma cruzi is expressed on the surface of E. coli, it induces the bacteria to undergo endocytosis in non-phagocytic animal cells and reach cytosol (Ortega-Barria et. al., supra). Expanding on this theme, it is feasible to identify and select high affinity antibodies that bind to specific receptors in cell lines and trigger endocytosis by constructing non-immunogenic phage or bacterial display libraries.

Specifically, PenetraBodies may be derived from HuMAb libraries. Accordingly, provided are antibody bacterial display libraries. A library preferably comprises a plurality of bacteria wherein the bacterial display, on average, has at least one copy of a scFv or V_{H} or V_{L}; the library comprises a plurality of species of scFv or V_{H} or V_{L}. In preferred embodiments, the bacterial display, on average, comprises at least 3, at least 4, or at least 5 copies of a scFv or V_{H} or V_{L} per bacterium. Particularly preferred libraries comprise, on average, at least about 10⁶, preferably at least about 10⁷, more preferably at least about 10⁸ different species of scFv or V_{H} or V_{L}. In a most preferred embodiment, the antibodies are encoded by a nucleic acid that is part of plasmid or phagemid vectors. In still yet another embodiment, this invention provides a nucleic acid library encoding as the bacterial display antibody libraries. The nucleic acid library comprises at least about 10⁶, more preferably at least about 10⁷, and most preferably at least about 10⁸ different plasmid or phagemid vectors.

Endocytosed bacteria can be selected by two different methods. One way is to lyse and plate these mammalian cells on bacterial media containing appropriate antibiotic markers but such procedures are cumbersome and laborious when a large library of >10⁸ variants has to be screened. Another approach is to express a fluorescent protein such as GFP in E. coli, and once endocytosed, the mammalian cell is fluorescent and can be isolated by FACS. GFP is a novel fluorescent marker to select for bacteria that are endocytosed because of the following features: a) GFP is a cytoplasmic protein with low toxicity (Chalfie et. al., Science 263:802, 1994); therefore, the presence of GFP should have minimal effects on the bacterial cell surface dynamics; b) GFP can be continuously synthesized, which minimizes the effect of fluorescence-signal dilution during bacterial replication; and c) GFP is easily imaged and quantitated (Wang and Hazelrigg, Nature 369:400, 1994). Furthermore, the fluorescence intensity of a single mammalian cell is directly proportional to the number of bacteria associated with it (Valdivia et. al., Gene 173:47, 1996). Therefore, flow cytometric analysis of GFP-producing bacteria associated with host cells provides a rapid and convenient measurement of bacterial adherence and invasion. It has been shown that a) the gene gfp is expressed and a functional fluorescent GFP is produced in diverse bacterial systems such as E. coli, Yersinia pseudotuberculosis, Salmonella typhimurium, and Mycobacterium marinum, b) production of GFP did not alter the interaction of three pathogens with their respective host cells, c) intracellular bacterial pathogens producing GFP can be imaged in association with live cells and tissues, and d) GFP production can be detected by flow cytometry and be used to measure the degree of bacterial association with mammalian cells (Valdivia et. al. supra).

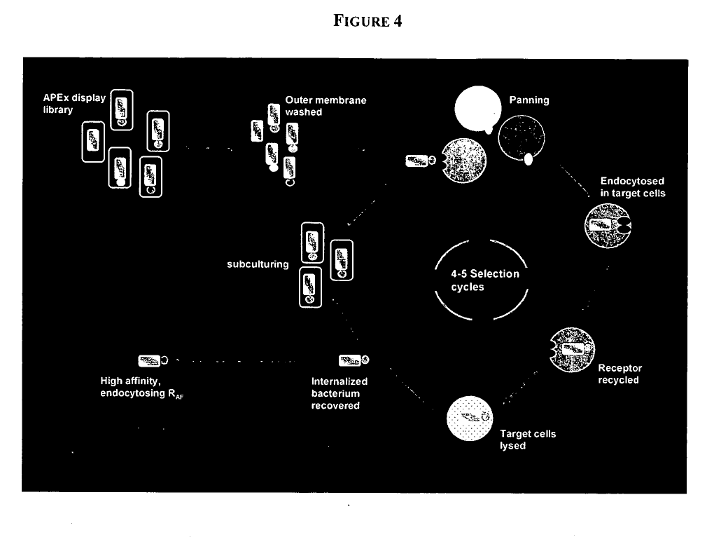

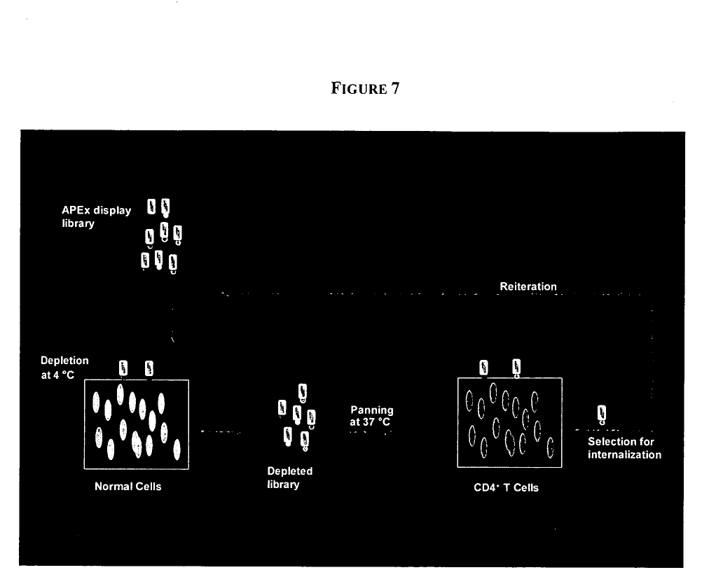

It is possible to directly select internalizing antibodies (R_{AF}) from large non-immune or immune bacterial display libraries by recovering internalized bacteria from within mammalian cells after receptor-mediated endocytosis (FIGS. 4, 7). Thus, in one embodiment, this invention provides methods of selecting polypeptide or antibody binding R_{AF} domains that are internalized into specific target cells. The methods involve a) contacting one or more of target cells with one or more members of a bacterial display library; b) culturing the target cells under conditions where members of the display library can be internalized; and c) identifying internalized members of the bacterial display library if members of the bacterial display library are internalized into one or more of the target cells. Preferably, the methods additionally involve contacting members of the bacterial display library with cells of subtractive cell lines; and then washing the target cells to remove the cells of a subtractive cell line; and to remove members of the bacterial display library that are non-specifically bound or weakly bound to the target cells. In a preferred embodiment, the bacterial display library is an antibody bacterial display library, more preferably an antibody bacterial display library displaying single chain antibodies (scFv), or the variable domains of either light (V_{L}) or heavy (V_{H}) chains.

In a preferred embodiment, the identifying step comprises recovering internalized bacterium and repeating steps of the process again to further select for internalizing binding moieties. In one embodiment, the recovering step involves lysing the target cells to release internalized bacterium, and sub-culturing the bacterium to produce bacteria for a subsequent round of selection. The recovering step can involve recovering infective bacterium, and/or recovering a nucleic acid encoding a bacterial-displayed antibody and/or selection of bacteria expressing a selectable marker. The identifying step can involve detecting expression of a reporter gene, detecting the presence or quantity of a particular nucleic acid, or selection of bacterium via a selectable marker. The identifying step can also involve sorting of mammalian cells with internalized bacteria by FACS. In preferred methods, the cells of a subtractive cell line are present in at least 2-fold excess over the target cells. In preferred methods, the target cell line is grown adherent to a tissue culture plate and co-incubated with the subtracting cell line in suspension in a single culture flask. In particularly preferred methods, the contacting with a subtractive cell line is performed at a temperature (for example, at 4° C.) lower than the internalization culture conditions (for example, at 37° C.). In particularly preferred embodiments, the bacteria express a selectable marker and/or a reporter gene. Preferred selectable markers include, but are not limited to genes (or cDNAs) encoding fluorescent proteins (for example, GFP), and a chromogenic gene or cDNA (for example, beta lactamase, luciferase, and beta galactosidase). In certain embodiments, the target cells can include cells that over express a particular receptor, members of a cDNA expression library, cells that over express a chemokine receptor, cells of a transformed cell line, cells transformed with a gene or cDNA encoding a specific surface target receptor. Suitable subtractive cell lines include, but are not limited to normal human fibroblasts, normal human breast cells, pancreatic cells, and cardiomyocytes.

The cell-surface receptors involved in receptor-mediated endocytosis can be identified de novo (Gao et. al., J. Immunol. Meth. 274:185, 2003). In the first step, through a subtractive approach, the tumor-specific internalizing scFvs are isolated by sequentially exposing the scFv library to various human cells and then finally to the desired cell type. As the next step, the selected scFvs are used as probes for the subsequent identification of their cognate receptors by immunoprecipitation, mass spectrometry and database searching. Based on this procedures scFvs specific to transferrin receptor in prostate tumor cells, and α₃β₁ integrin present in pancreatic adenocarcinoma cells were selected (Gao et. al., supra). Such a subtractive approach has been successfully used to select internalizing receptors on human breast and pancreatic carcinoma cell lines (Fransson et. al., Cancer Lett. 208:235, 2004) as well as on prostate carcinoma cells (Liu et. al., Cancer Res. 64:704, 2004).

Accordingly, the methods of this invention may also be used to identify internalizing receptors. Identifying an endocytosing receptor present only in hepatocytes (liver cells) and not in any other cell types is one such example. The methods generally involve any of the methods for identifying internalizing antibodies or polypeptides identified are used to probe the original target cells, or different cells. As the internalizing antibodies or polypeptides so bind, they permit isolation of the cell bearing the internalizing receptor and isolation of the receptor and/or the receptor epitope itself. Thus, in one embodiment the methods involve a) contacting one or more of the target cells with one or more members of a bacterial display library, b) optionally, but preferably, contacting members of the bacterial display library with cells of a subtractive cell line, c) optionally, but preferably, washing the target cells to remove said cells of a subtractive cell line and to remove members of the bacterial display library that are non-specifically bound or weakly bound or weakly bound to said target cells, d) culturing the cells under conditions where members of said bacterial display library can be internalized if bound to an internalizing marker, e) identifying internalized members of the bacterial display library if members of the bacterial display library are internalized into one or more of said target cells, f) contacting the same or different target cells with the identified internalized members of step (e) or members propagated therefrom, whereby the members bind to the surface of said target cells. The method can further involve isolating a component of the same or different target cells to which the members bind. In some methods the “identifying” step involves recovering internalized bacteria and repeating steps (a-e) to further select for internalizing receptors. The contacting, washing, culturing, and identifying steps are preferably performed as described herein, and the subtractive cell lines include cardiomyocytes, normal and cancerous breast cells. For example, human hepatocytes and B cells are the target cells in experiments intended to identify internalizing receptors to which the HCV-R_{AF} domain targets. Similarly, CD4⁺ T cells and macrophages are the target cells in experiments intended to identify internalizing receptors to which the HIV-R_{AF} domain targets.

In still yet another embodiment, this invention provides methods of selecting polypeptide or antibody binding moieties that target intracellular epitopes (I_{AF}). The methods involve a) contacting the labeled intracellular epitope with one or more members of a bacterial display library wherein the said binding occurs in the periplasmic space as the “intracellular” epitope is also targeted to periplasm; b) identifying the high affinity antibody (I_{AF}) domain from the bacterial display library, and then expressing the I_{AF} domain in bacterial cytoplasm along with the epitope; c) through directed evolution procedure coupled with FACS, making the I_{AF} domain soluble and functional in cytosol; and d) making the I_{AF} domain serum stable by mutating the destabilizing residues of the I_{AF}.

Other protein display technologies may be used in the above-described methods. Modification of such methods to incorporate other display technologies is well known to one of skill in the art. A review of exemplary protein display technologies that may be used in the present methods is provided below.

Protein Display Technologies:

Antibody engineering plays a critical role in developing antibody therapies with superior pharmacokinetic and pharmacodynamic properties (Burks et. al., Proc. Natl. Acad. Sci. USA 94:412, 1997; U.S. Pat. No. 6,180,341). Directed evolution involves, first, the generation of a recombinant library of protein-expressing clones with randomized sequences using molecular biology techniques, and second, the use of screening technologies for the isolation of protein variants that exhibit the most enhanced activity. The screening of large libraries requires a physical link among a gene, the protein it encodes, and the desired function. Such a link can be established by using a variety of in vivo display technologies that have proven to be invaluable (Wittrup, Nature Biotechnol. 18:1039, 2000; Hayhurst and Georgiou, Curr. Opin. Chem. Biol. 5:683, 2001).

Protein display technologies collectively represent one of the most powerful tools for protein engineering (Olsen et. al., Curr. Opin. Biotechnol. 11:331, 2000). For display purposes, a protein is fused to the C or N terminus of a polypeptide sequence that targets the resulting chimera onto the surfaces of biological particles such as viruses, bacteria, and yeast. Libraries are typically screened for ligand binding by a series of adsorption-desorption cycles by a process called “panning”. Panning has been used successfully to screen highly complex libraries made by cloning the mammalian antibody repertoire and displaying it on phage (up to 1011 clones). For somewhat less diverse libraries (up to 10⁹ clones), display on bacteria or yeast coupled with flow cytometry is a powerful tool for the discovery of proteins with exceptionally high ligand-binding affinities (Chen et. al., Nature Biotechnol. 19:537, 2001). Although the importance of display technologies for protein engineering is undisputed, the need to anchor the target polypeptide onto the surface of a biological particle imposes a number of limitations that can significantly reduce the diversity of the library relative to the totality of proteins that can be produced in a soluble form within the cell. First, protein display requires that the protein of interest be expressed as either a C- or N-terminal fusion, a process that can adversely affect protein function and/or stability. Second, protein display is subject to biological constraints associated with protein export and presentation, which may compromise the viability of the virus or cell. Third, display can introduce screening artifacts such as avidity effects in phages (O'Connell et. al., J. Mol. Biol. 321:49, 2002).

A: Phage Display Library:

Display on M13 bacteriophage is the oldest and the most widely used protein library-screening method (Marks et. al., J. Mol. Biol. 222:581, 1991; Marks et. al., J. Biol. Chem. 267:16007, 1992; Rodi and Makowski, Curr. Opin. Biotechnol. 10:87, 1999). Phage antibody libraries have become an important resource for the development of therapeutic antibodies (Bradbury and Marks, J. Immunol. Meth. 290:29, 2004). Large non-immune libraries serve as a single pot resource for the rapid generation of human MAbs (HuMAbs) to a wide range of self and non-self antigens, including tumor growth factor receptors (Li et. al., Cancer Gene Ther. 8:555, 2001; Liu et. al., Cancer Res. 64:704, 2004). Most of the MAbs isolated from combinatorial libraries expressed on phage have been selected using purified antigens or peptides immobilized on artificial surfaces. This approach may select MAbs that do not recognize the native protein in a physiological context, especially with large molecular mass cell surface receptors. Attempts have been made to select antigen in native conformation using either cell lysates (Parren et. al., J. Virol. 70:9046, 1996; Sanna et. al., Proc. Natl. Acad. Sci. USA 92:6439, 1995; Sawyer et. al., J. Immunol. Meth. 204:193, 1997) or living cells (Andersen et. al., Proc. Natl. Acad. Sci. USA 93:1820, 1996; Osbourn et. al., Immunotechnol. 3:293, 1998). Because of the heterogeneity of the starting material, such approaches require elaborate protocols including subtractive steps to avoid the selection of irrelevant antibodies. The few successful selections performed on heterogenous material were generally done using small libraries from immunized sources. The use of immunized libraries limits the spectrum of antigen specificities that can potentially be obtained from the same library and typically yield murine antibodies. There are only three reports of successful selection on cells using large non-immune libraries (de Kruif et. al., Proc. Natl. Acad. Sci. USA 92:3938, 1995; Marks et. al., Biotechnology 11:1145, 1993; Vaughan et. al., Nature Biotechnol. 14:309, 1996).

The step limiting the selection of binders from large naïve libraries by cell panning seems to be the relatively high background binding of non-specific phage and relatively low binding of specific phage (Becerril et. al., Biochem. Biophys. Res. Comm. 255:386, 1999; Pereira et. al., J. Immunol. Meth. 203:11, 1997; Watters et. al., Immunotechnol. 3:21, 1997). The low binding of specific phage is partially related to the low concentration of a given binding phage in the polyclonal preparation (approximately 1.6×10⁻¹⁷ M for a single member of a 10⁹ library in a phage preparation of 1×10¹³ particles/ml). The low concentration simultaneously limits the efficiency of both subtraction of common binders and enrichment of specific binders. To overcome this limitation, it was resorted to take advantage of normal cell surface receptor biology. Many receptors undergo endocytosis upon ligand binding. It was hypothesized that enrichment ratios of specific binders could be significantly increased by recovering endocytosed phage antibodies from the cytosol after stringent removal of non-specific phage from the cell surface (Poul et. al., J. Mol. Biol. 301:1149, 2000).

B: Yeast Surface Display Library:

Yeast surface display (YSD; Boder and Wittrup, Nature Biotechnol. 15:553, 1997) is another proven tool for protein engineering. In YSD, the protein of interest is expressed as a fusion with a yeast mating protein, Aga2p, which is targeted to the yeast cell wall. Once expressed on the yeast surface, protein properties such as stability and affinity, can be quantitatively measured using fluorescently labeled reagents and flow cytometry. Further, libraries of mutants can be sorted for desired properties using fluorescent activated cell sorting (FACS). YSD has been successfully applied to several facets of antibody engineering: isolation of novel Abs against specific antigens from a non-immune HuMAb library (Feldhaus et. al., Nature Biotechnol. 21:163, 2003); affinity maturation resulting in the highest affinity antibody reported to date (Boder et. al., Proc. Natl. Acad. Sci. USA 97:10701, 2000); and stability and extracellular expression optimization (Shusta et. al., Nature Biotechnol. 18:754, 2000). In addition, YSD is a useful tool for domain-level analysis of an antibody's binding site (paratope), and engineering of functional intrabodies (Colby et. al., J. Mol. Biol. 342:901, 2004). In an attempt to identify a minimal antibody fragment with superior expression and intracellular function, YSD was used to engineer an intracellularly non-functional scFv into a functional single-domain V_{L} intrabody through affinity maturation and binding site analysis.

Not withstanding all these advantages, a potential shortcoming of the YSD platform for application to intrabody engineering might arise from the difference in redox environment on the cell surface as compared to the cytoplasm, where disulfide bonds do not stably form. MAbs contain highly conserved intradomain disulfide bonds in both the V_{H} and V_{L} domains that hold the β-sheet-forming framework residues in a rigid conformation. Disruption of these disulfide bonds perturbs the domain structure, reducing protein stability (Ramm et. al., J. Mol. Biol. 290:535, 1999). This presumably is responsible for the disparity between cell surface expression and cytoplasmic expression levels for the scFv. Further, the expression of fusion proteins (for example, scFv) is generally cis-dominant; that is, the expression of the fusion protein is only as good as the expression of the member with the lowest stability, so an alternative explanation of the improvement in expression observed when the V_{H} is eliminated is that the V_{H} domain of 2.4.3 was significantly less stable than the V_{L} under reducing conditions (Colby et. al., supra).

An important issue with any library screening technology (both phage and yeast display technologies) is the ability to express isolated clones at a high level. Existing display formats involve fusion to large anchoring sequences, which can influence the expression characteristics of the displayed proteins. For this reason, scFvs that display well as fusions in phage, yeast, or bacteria (particularly the protein libraries expressed on the outer membrane) may not necessarily be amenable to high expression in soluble form as nonfusion proteins (Hayhurst et. al., J. Immunol. Meth. 276:185, 2003). In contrast, the short (6-aa) sequence required for N-terminal tethering of proteins onto the cytoplasmic membrane in APEx display is unlikely to affect the expression characteristics of the fusion. Consistent with this hypothesis, all three affinity-enhanced clones to the anthrax PA toxin isolated by APEx exhibited excellent soluble expression characteristics despite having numerous amino acid substitutions, suggesting that the isolation of clones that can readily be produced in soluble form in bacteria on a large scale may be an intrinsic feature of APEx selections (Harvey et. al., Proc. Natl. Acad. Sci. USA 101:9193, 2004).

C: APEx Bacterial Display Library:

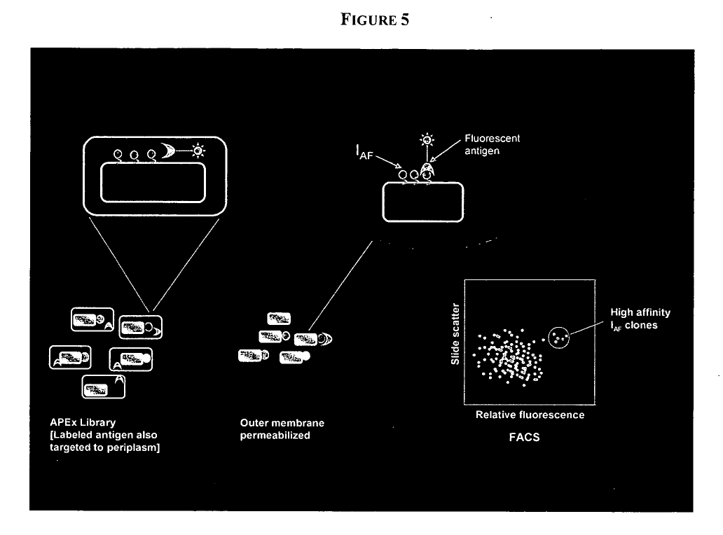

A flow cytometry-based method has been developed using bacterial expression for the efficient selection of high-affinity ligand-binding proteins, and specifically scFvs, from combinatorial libraries. APEx is based on the anchoring of proteins to the periplasmic side of the inner membrane, followed by disruption of the outer membrane before incubation with fluorescently labeled antigen and FC sorting (Harvey et. al., Proc. Natl. Acad. Sci. USA 101:9193, 2004). In APEx, proteins are expressed in periplasm by tethering to the inner membrane of E. coli. After chemical/enzymatic permeabilization of the bacterial outer membrane, E. coli cells expressing anchored scFv antibodies can be specifically labeled with fluorescent antigens, ranging in size up to at least 240 kDa, and analyzed by FC (FIGS. 4, 5). Another advantage is that fusions between GFP and antigen can be expressed endogenously and captured by periplasmically anchored scFv. Thus, after a washing step, cells that express both the fluorescent antigen and an APEx-anchored scFv are highly fluorescent and can be readily sorted from cells that express either only an scFv or GFP-antigen fusion alone.

With sorting rates of >400 million cells per hour, commercial FC machines can be used to screen libraries of the size accessible within the constraints of microbial transformation efficiencies. Furthermore, mutliparameter FC can provide valuable information regarding the function of each and every library clone in real time, thus helping to guide the library construction process and optimize sorting conditions (Daugherty, P. S. et. al., Proc. Natl. Acad. Sci. USA 97:2029, 2000). In particular, E. coli offers facile expression of recombinant protein high DNA transformation efficiencies that allow for efficient large library production and increased coverage of protein library sequence space.

APEx display offers several advantages over previously developed bacterial periplasmic expression with cytometric screening method, called PECS (Chen et. al., Nature Biotechnol. 19:537, 2001), as well as surface display approaches such as phage and yeast display technologies: (i) APEX is an E. coli based system and therefore provides an easy route to the creation of large libraries by transformation and preparative protein expression of isolated antibodies; (ii) by using a fatty acylated anchor to retain the protein in the inner membrane, a fusion as short as 6 amino acids is all that is required for display. The short fusion is unlikely to influence the affinity or expression characteristics of the isolated proteins; (iii) the inner membrane lacks molecules such as LPS or other complex carbohydrates that can sterically interfere with large antigen binding to displayed polypeptides; (iv) the fusion must only traverse one membrane before it is displayed, and therefore biosynthetic limitations that might restrict the export of certain sequences to the yeast or bacterial surface may be circumvented; (v) display is accomplished by using either N- or C-terminal fusion, (vi) APEx can be used directly for proteins expressed from widely used phage display vectors. Finally, (vii) APEx provides a means for the simultaneous expression of fluorescent antigen and antibodies within the same cell. This is particularly important for peptide antigens, and circumvents time-consuming processes for synthesis, purification, and conjugation of preparative amounts of probe, as is required when the fluorescent antigen is incubated with the library. APEx can be used for the detection of antigens ranging from small molecules (<1 kDa) to phycoerythrin conjugates (240 kDa), and possibly much larger antigens.

APEx display procedure can be used to derive a single domain antibodies (DAbs) from an scFv, when the binding energy of the scFv is contributed predominantly by one of the two domains. Reduction to a single domain can be advantageous for increasing intracellular expression levels, which is key to the biological activity of intrabodies; these procedures may also be useful for focusing further engineering efforts, such as affinity maturation, on the more relevant domain.

3. Exemplary Penetrabodies and Methods of Making Penetrabodies

PenetraBodies may comprise human antibody (HuMAb) fragments or recombinant of scFv, V_{H} or V_{L} domains. A disulfide bond may be introduced between the two domains of an scFv for extra stability. The original fragments for use within a particular PenetraBody may be selected from bacterial display libraries. The R_{AF} domain selectively targets a specific receptor on a particular cell, gets internalized through receptor-mediated endocytosis, and released into cytosol. The I_{AF} domain is the intrabody component of the PenetraBody and binds to the intended epitope in cytosol. The R_{AF} and I_{AF} domains are linked by a flexible linker, which could also function as an endosomal escape domain.

The antigen recognition sites or entire variable regions of the domains comprising the PenetraBodies may be derived from one or more parental antibodies directed against any antigen of interest. The parental antibodies can include naturally occurring antibodies or antibody fragments, antibodies or antibody fragments adapted from naturally occurring antibodies, antibodies constructed de novo using sequences of antibodies or antibody fragments known to be specific for an antigen of interest. Sequences that may be derived from parental antibodies include heavy and/or light chain variable regions and/or CDRs, framework regions or other portions thereof.

PenetraBodies Targeting HIV Proteins in Cytosol: In one embodiment, a PenetraBody may target CD4+ T cells and macrophages, get internalized, and target HIV proteins in cytosol. In this regard, such a PenetraBody may comprise a) an R_{AF} domain and b) an I_{AF} domain, wherein the R_{AF} domain binds to a specific receptor on hepatocytes, triggers endocytosis and enters cytosol, and then the I_{AF} domain binds to a highly conserved epitope of a HIV protein.

The reverse transcriptase (RT) of HIV-1 is crucial for the early stages of HIV life cycle and its inhibition is likely to lead to an abortive infection. RT copies the viral genomic single-stranded RNA into double stranded-DNA for subsequent integration into the cellular DNA by the viral enzyme, integrase. Blocking the HIV infection at this stage prevents the viral DNA synthesis and therefore the subsequent integration of the viral genome into the cellular DNA leading to an abortive infection (Shaheen et. al., J. Virol. 70:3392, 1996). A number of murine antibodies targeted against RT was previously isolated, and when expressed inside human cells, efficiently blocked HIV replication (Shaheen et. al., supra; Maciejewski et. al., Nature Med. 1:667, 1995). Given that the murine or humanized MAbs elicit in vivo immune reactions, the best approach is to develop HuMAbs. Another key consideration is that the MAbs be specific enough to RT but not to human DNA polymerases in the cell (Gargano, et. al., J. Virol. 70:7706, 1996). Human scFvs specifically inhibiting the RNA-dependent DNA polymerase and DNA dependent DNA polymerase activity of RT were isolated (Herschhorn et. al., Biochim. Biophys. Acta 1648:154, 2003). One of the scFvs, F-6, that inhibits both of the activities did in fact bind to the carboxy terminus of the large subunit of RT, which contains the ribonuclease H domain, and not the polymerase domain of the protein. The scFvs were extremely specific to the HIV-RT and did not inhibit other RTs tested. Further it was shown that the V_{H}-CDR3 peptide itself inhibited the RDDP activity (Herschhorn et. al., supra). Such scFvs and fragments of these monoclonal antibodies may comprise an I_{AF} domain of a PenetraBody.

One of the critical determinants of HIV-1 infectivity in vivo is the Vif (virion infectivity factor) protein, which is essential for the establishment of productive infection in peripheral blood lymphocytes and for pathogenesis in animal models of AIDS (Chowdhury et. al., J. Virol. 70:5336, 1996). Vifacts during late steps of the viral life cycle to increase the infectivity of HIV-1 by as much as 100-1000 fold most likely by affecting virus assembly or maturation (Öhagen and Gabuzda, J. Virol. 74:11055, 2000), and plays a critical role in the cell-free and cell-to-cell transmissions of HIV-1 (Gabuzda et. al., J. Virol. 66:6489, 1992; von Schwedler et. al., J. Virol. 67:4945, 1993). Vif-specific scFv when expressed intracellularly in the cytoplasm bound the Vif protein and neutralized its function. Cells expressing Vif-intrabody were highly refractory to challenge with different strains of HIV-1 (Goncalves et. al., J. Biol. Chem. 277:32036, 2002). Such scFvs may comprise an I_{AF} domain of a PenetraBody.

PenetraBodies Targeting HCV Proteins in Cytosol: In another embodiment, a PenetraBody may target hepatocytes and B cells, get internalized, and target HCV proteins in cytosol. A PenetraBody may therefore comprise a) an R_{AF} domain and b) an I_{AF} domain, wherein the R_{AF} domain binds to a specific receptor on hepatocytes, triggers endocytosis and enters cytosol, and then the I_{AF} domain binds to a highly conserved epitope of a HCV protein.

The viral genome of HCV is approximately 9600 nucleotides (nts) long, and consists of a highly conserved 5′ untranslated region (UTR), a single long open reading frame (ORF) of approximately 9,000 nts and a complex 3′ UTR. The ORF encodes a large polypeptide precursor that is cleaved into at least ten proteins by host and viral proteases. The structural proteins C (core), E1, and E2 (envelope glycoproteins) are contained at the N-terminus of the polyprotein and are followed by p7 and the nonstructural proteins (NS) 2, 3, 4A, 4B, 5A and 5B.

The NS3 protease is a multidomain 70 kDa protein that has protease domain at the N-terminus and RNA helicase domain at its C-terminus (Kime et. al., Biochem. Biophys. Res. Comm. 215:160, 1995). Most precursor proteolytic processing events are directed by the virally encoded NS3 serine protease, and it is essential for HCV replication (Failla et. al., J. Virol. 68:3753, 1994). In addition, NS3 has been found to interfere with signal transduction pathways (Borowski et. al., Arch. Virol. 144:687, 1999), and promote cell proliferation and cell transformation (Zemel et. al., J. Viral Hepatol. 8:96, 2001).

The non-structural protein, NS5B, is the RNA-dependent RNA polymerase (RdRp) and is shown to be crucial for viral replication in vivo (Kolykhalov et. al., J. Virol. 74:2046, 2000). A murine MAb (5B-12B7) as well as its scFv that inhibits the HCV RdRp has been isolated (Moradpour et. al., J. Biol. Chem. 277:593, 2002). The MAb binds to a conformational epitope of the palm subdomain of the RdRp. The inhibition was specific to the HCV RdRp and did not inhibit swine fever virus NS5B and poliovirus 3D polymerase.

Thus, the scFvs and fragments of antibodies directed against such HCV proteins or fragments thereof may comprise an I_{AF} domain of a PenetraBody.

In yet another embodiments, fragments of antibodies directed against proteins expressed in breast cancer or fragments thereof (for example, Her2) may comprise an I_{AF} domain of a PenetraBody. A fragment of Herceptin® (trastuzumab) may be used, for example.

Variants of a given PenetraBody may be obtained by any suitable method, including those methods discussed herein. Preferably, the variants of the PenetraBodies contain from 1 to about 30 amino acid substitutions, deletions, inversions, and/or insertions thereof. The PenetraBodies preferably recognize one or more strains of HCV and HIV or other infectious agent. Preferably, the molecules are broadly cross-reactive and can bind to a wide range of isolates of HIV and HCV or ther clinical isolates from different genotypes with high affinity.

Functional or active regions of the PenetraBody may be identified and/or improved by mutagenesis of a specific region of the protein, followed by expression and testing of the expressed polypeptide. For example, amino acid sequence variants of antibodies or antibody fragments can be generated and those that display equivalent or improved affinity for antigen can be identified using standard techniques and/or those described herein. Methods for generating amino acid sequence variants are readily apparent to a skilled practitioner in the art and can include directed evolution technologies (U.S. Pat. No. 6,180,341) or random mutagenesis (e.g., by PCR) of the nucleic acid encoding the PenetraBody (Zoller, M. J. Curr. Opinion in Biotechnol. 3:348-354, 1992). Both naturally occurring and non-naturally occurring amino acids may be used to generate amino acid sequence variants of the PenetraBodies.

In vitro scanning saturation mutagenesis is a particularly powerful and rapid way of identifying mutants with interesting properties that can be produced in large quantities and subjected to more detailed structural and functional characterization. SSM systematically explores in vitro antibody affinity evolution, analogous to somatic hypermutation in vivo. Interesting single mutants can be used as a starting point for subsequent rounds of SSM at other sites, so that multiple mutations with synergistic effects on binding may be identified. This same sequential mutation approach should be useful to optimize properties such as affinity, potency, efficacy, altered specificity, reduced immunogenicity, and removal of proteolytic cleavage sites (Burks et. al., Proc. Natl. Acad. Sci. USA 94:412, 1997; Chen et. al., Prot. Engg. 12:349, 1999; U.S. Pat. No. 6,180,341).

The substitutions, deletions, inversions, and/or insertions of the PenetraBody preferably occur in non-essential regions. The identification of essential and non-essential amino acids in PenetraBody can be achieved by methods known in the art, such as by site-directed mutagenesis (for example, SSM) and AlaScan analysis (Moffison et. al., Chem. Biol. 5:302-307, 2001). Essential amino acids have to be maintained or replaced by conservative substitutions in the variants of the R_{AF} or I_{AF} domains. Non-essential amino acids can be deleted, or replaced by a spacer or by conservative or non-conservative substitutions.

The variants can be obtained by substitution of any of the amino acids present in the PenetraBody. As can be appreciated, there are positions in the sequence that are more tolerant to substitutions than others, and some substitutions can improve the binding activity of the PenetraBody. The amino acids that are essential should either be identical to the amino acids present in the PenetraBody, or substituted by conservative substitutions. The amino acids that are non-essential can be identical to those in the PenetraBody, can be substituted by conservative or non-conservative substitutions, and/or can be deleted.

The parental human monoclonal antibodies comprising the PenetraBodies can be prepared using any technique. Examples of techniques for human monoclonal antibody production include those described by Cole et al., (Monoclonal Antibodies and Cancer Therapy, Alan R. Liss, p. 77, 1985) and by Boemer et al., (J. Immunol., 147:86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991; and C. F. Barbas, D. R. Burton, J. K. Scott, G. J. Silverman, Phage Display: A Laboratory Manual, Cold Spring Harbor Laboratory Press, Cold Spring Harbor, N.Y., 2001), yeast surface display libraries (Boder and Wittrup, Nature Biotechnol. 15:553, 1997; Feldhaus et. al., Nature Biotechnol. 21:163, 2003), and bacterial display libraries (Harvey et. al., Proc. Natl. Acad. Sci. USA 101:9193, 2004; Wernerus et. al., Appl. Environ. Microbiol. 69:5328, 2003).

Any of the antibody portions of the PenetraBodies also may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Candidate antibodies for inclusion in the PenetraBodies, or the PenetraBodies themselves, may be screened for activity using a variety of known assays. For example, screening assays to determine binding specificity are well known and routinely practiced in the art. For a comprehensive discussion of such assays, see Harlow et al. (Eds.), ANTIBODIES: A LABORATORY MANUAL; Cold Spring Harbor Laboratory; Cold Spring Harbor, N.Y., 1988, Chapter 6.

In certain embodiments, the domains comprising a PenetraBody are produced separately and then linked, e.g. covalently linked, to each other. For example, a R_{AF} and I_{AF} domain are produced separately in vitro, purified, and mixed together under conditions under which the tag will be able to be linked to the polypeptide of interest. For example, the domains can be obtained (isolated) from a source in which it is known to occur, can be produced and harvested from cell cultures, can be produced by cloning and expressing a gene encoding the desired domain, or can be synthesized chemically. Furthermore, a nucleic acid sequence encoding the desired domain can be synthesized chemically.

Linkers (also known as “linker molecules” or “cross-linkers”) may be used to conjugate domains. Linkers include chemicals able to react with a defined chemical group of several, usually two, molecules and thus conjugate them. The majority of known cross-linkers react with amine, carboxyl, and sulfhydryl groups. The choice of target chemical group is crucial if the group may be involved in the biological activity of the polypeptides to be conjugated. For example, maleimides, which react with sulfhydryl groups, may inactivate Cys-comprising peptides or proteins that require the Cys to bind to a target. Linkers may be homofunctional (comprising reactive groups of the same type), heterofunctional (comprising different reactive groups), or photoreactive (comprising groups that become reactive on illumination).

Linker molecules may be responsible for different properties of the conjugated compositions. The length of the linker should be considered in light of molecular flexibility during the conjugation step, and the availability of the conjugated molecule for its target (cell surface molecules and the like.) Longer linkers may thus improve the biological activity of the compositions of the present invention, as well as the ease of preparation of them. The geometry of the linker may be used to orient a molecule for optimal reaction with a target. A linker with flexible geometry may allow the cross-linked polypeptides to conformationally adapt as they bind other polypeptides. The nature of the linker may be altered for other various purposes. For example, the aryl-structure of MBuS was found less immunogenic than the aromatic spacer of MBS. Furthermore, the hydrophobicity and functionality of the linker molecules may be controlled by the physical properties of component molecules. For example, the hydrophobicity of a polymeric linker may be controlled by the order of monomeric units along the polymer, e.g. a block polymer in which there is a block of hydrophobic monomers interspersed with a block of hydrophilic monomers.

The chemistry of preparing and utilizing a wide variety of molecular linkers is well-known in the art and many pre-made linkers for use in conjugating molecules are commercially available from vendors such as Pierce Chemical Co., Roche Molecular Biochemicals, United States Biological, and the like.

A PenetraBody may also be produced by recombinant means. For example, a nucleic acid encoding the R_{AF} domain can be joined to either end of a nucleic acid sequence encoding the I_{AF} domain such that the two protein-coding sequences are sharing a common translational reading frame and can be expressed as a fusion protein including both domains. The combined sequence is inserted into a suitable vector chosen based on the expression features desired and the nature of the host cell. Following expression in the chosen host cell, fusion protein can be purified by routine biochemical separation techniques or by immunoaffinity methods using an antibody to one or the other part of the fusion protein. Alternatively, the selected vector can add a tag to the fusion protein sequence, e.g., an oligohistidine tag as described in the examples presented hereinafter, permitting expression of a tagged fusion protein that can be purified by affinity methods using an antibody or other material having an appropriately high affinity for the tag. Sambrook et al., Molecular Cloning: A Laboratory Manual, 2d Ed., Cold Spring Harbor Laboratory Press (1989); Deutscher, M. Guide to Protein Purification Methods Enzymology, vol. 182. Academic Press, Inc. San Diego, Calif. (1990). If a vector suitable for expression in mammalian cells is used. e.g., one of the vectors discussed below, the fusion protein can be expressed and purified from mammalian cells. Alternatively, the mammalian expression vector (including fusion protein-coding sequences) can be administered to a subject to direct expression of a protein fusion polypeptide in the subject's cells. A nucleic acid encoding a fusion polypeptide can also be produced chemically and then inserted into a suitable vector for fusion protein production and purification or administration to a subject. Finally, a fusion protein can also be prepared chemically.

Techniques for making fusion genes are well known in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992). Accordingly, provided is an isolated nucleic acid comprising a fusion gene of a gene encoding at least one R_{AF} domain and one I_{AF} domain.

The nucleic acid may be provided in a vector comprising a nucleotide sequence encoding a fusion polypeptide, and operably linked to at least one regulatory sequence. It should be understood that the design of the expression vector may depend on such factors as the choice of the host cell to be transformed and/or the type of protein desired to be expressed. The vector's copy number, the ability to control that copy number and the expression of any other protein encoded by the vector, such as antibiotic markers, should be considered. Such vectors may be administered in any biologically effective carrier, e.g., any formulation or composition capable of effectively transfecting cells either ex vivo or in vivo with genetic material encoding a chimeric polypeptide. Approaches include insertion of the nucleic acid in viral vectors including recombinant retroviruses, adenoviruses, adeno-associated viruses, human immunodeficiency viruses, and herpes simplex viruses-1, or recombinant bacterial or eukaryotic plasmids. Viral vectors may be used to transfect cells directly; plasmid DNA may be delivered alone with the help of, for example, cationic liposomes (lipofectin) or derivatized (e.g., antibody conjugated), polylysine conjugates, gramicidin S, artificial viral envelopes or other such intracellular carriers. Nucleic acids may also be directly injected. Alternatively, calcium phosphate precipitation may be carried out to facilitate entry of a nucleic acid into a cell.

The subject nucleic acids may be used to cause expression and over-expression of a fusion polypeptide in cells propagated in culture, e.g. to produce fusion proteins or polypeptides.

Provided also is a host cell transfected with a recombinant gene in order to express a fusion polypeptide. The host cell may be any prokaryotic or eukaryotic cell. For example, a fusion polypeptide may be expressed in bacterial cells, such as E. coli, insect cells (baculovirus), yeast, insect, plant, or mammalian cells. In those instances when the host cell is human, it may or may not be in a live subject. Other suitable host cells are known to those skilled in the art. Additionally, the host cell may be supplemented with tRNA molecules not typically found in the host so as to optimize expression of the polypeptide. Other methods suitable for maximizing expression of the fusion polypeptide will be known to those in the art.

A cell culture includes host cells, media and other byproducts. Suitable media for cell culture are well known in the art. A fusion polypeptide may be secreted and isolated from a mixture of cells and medium comprising the polypeptide. Alternatively, a fusion polypeptide may be retained cytoplasmically and the cells harvested, lysed and the protein isolated. A fusion polypeptide may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of a fusion.

Thus, a nucleotide sequence encoding all or part of a fusion polypeptide may be used to produce a recombinant form of a protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant fusion polypeptides by microbial means or tissue-culture technology in accord with the subject invention.

The PenetraBodies produced as described above may be purified to a suitable purity for use as a pharmaceutical composition. Generally, a purified composition will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a PenetraBody using standard techniques for protein purification, for example, immunoaffinity chromatography, size exclusion chromatography, etc. in light of the teachings herein. Purity of a polypeptide may be determined by a number of methods known to those of skill in the art, including for example, amino-terminal amino acid sequence analysis, gel electrophoresis and mass-spectrometry analysis.

4. Therapeutic Methods and Pharmaceutical Compositions

Comprehended by the invention are pharmaceutical compositions comprising an effective amount of a PenetraBody, optionally together with pharmaceutically acceptable diluents, preservatives, solubilizers, emulsifiers, adjuvants and/or carriers needed for administration. The optimal pharmaceutical formulation for a desired biologically active agent will be determined by one skilled in the art depending upon the route of administration and desired dosage.

Antibodies of the invention are preferably administered to a subject in a pharmaceutically acceptable carrier. Suitable carriers and their formulations are described elsewhere (Remington: The Science and Practice of Pharmacy [19th ed.] ed. A. R. Gennaro, Mack Publishing Company, Easton, Pa. 1995). Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7.0 to about 7.5. It will be apparent to those persons skilled in the art that certain carriers may be more preferable depending upon, for instance, the route of administration and concentration of antibody being administered.

One skilled in the art will be able to ascertain effective dosages by administration and observing the desired therapeutic effect. Preferably, the formulation of the PenetraBody will be such that between about 0.01 μg PenetraBody/kg body weight/day and about 10 mg PenetraBody/kg body weight/day will yield the desired therapeutic effect. The effective dosages may be determined using diagnostic tools over time. The therapeutic dosages are determined depending on the severity of the infection, viral load (sustained virological response) over the course of therapy. The dosages may therefore vary over the course of therapy, with, for example, a relatively high dosage being used initially, until therapeutic benefit is seen, and lower dosages used to maintain the therapeutic benefits. Thus, effective dosages and schedules for administering the antibodies may be determined empirically, and making such determinations is within the skill in the art. Those skilled in the art will understand that the dosage of antibodies that must be administered will vary depending on, for example, the subject that will receive the antibody, the route of administration, particular type of antibody used and other drugs being administered.

In another aspect, in certain embodiments, the compounds may be administered as such or in admixtures with pharmaceutically acceptable carriers and may also be administered in conjunction with other agents. Conjunctive (combination) therapy thus includes sequential, simultaneous and separate, or co-administration of the active compound in a way that the therapeutic effects of the first administered one has not entirely disappeared when the subsequent is administered.

The present invention also relates to methods of treating individuals having an infection with PenetraBodies. Upon administering the PenetraBody to humans in need thereof, it selectively targets to a particular receptor on specific cells and gets rapidly internalized into cytosol, binds to an internal epitope and arrests the activity of the protein, whereupon the progression of infection/disease is inhibited or neutralized. In one embodiment, a method of inhibiting chronic HIV and HCV infections in humans is provided, which method comprises administering to humans in need thereof a PenetraBody comprising (a) scFv or the V_{H} or V_{L} domains, collectively called as the R_{AF} component wherein the R_{AF} domain selectively and preferentially binds to a receptor, gets internalized and delivered into cytosol, and (b) scFv or the V_{H} or V_{L} domains, collectively called as the I_{AF} component wherein the I_{AF} domain binds to a highly conserved epitope of a HIV or HCV protein in cytosol, whereupon the HIV or HCV infection is inhibited.

The antibodies can be administered to the subject or patient by injection (e.g., but not limited to, intravenous, intradermal, subcutaneous, intramuscular), or by other methods such as infusion that ensures its delivery to the bloodstream in an effective form.

Following administration of an antibody for treating, inhibiting, or preventing HIV or HCV infection, the efficacy of the therapeutic antibody can be assessed in various ways well known to the skilled practitioner. For instance, one of ordinary skill in the art will understand that an antibody of the invention is efficacious in treating or inhibiting HIV or HCV infections in a subject by observing that the antibody reduces viral load or delays or prevents a further increase in viral load. Viral loads can be measured by methods that are known in the art, for example, using PCR assays to detect the presence of HCV nucleic acid or antibody assays to detect the presence of HIV or HCV protein in a sample (e.g., but not limited to, blood or another body fluid) from a subject or patient, or by measuring the level of circulating anti-HIV or HCV antibodies in the patient. An antibody treatment that decreases HIV or HCV viral load in a HIV or HCV-positive patient is considered an efficacious antibody treatment.

In subjects who have been newly exposed to HIV or HCV but who have not yet displayed the presence of the virus (as measured by PCR or other assays for detecting the virus) in blood or other body fluid(s), efficacious treatment with an antibody of the invention partially completely inhibits or delays the appearance of the virus or minimizes the level of the virus in the blood or other body fluid(s) of the exposed individual.

5. Kits

The present invention provides kits for selecting and/or producing a PenetraBody. Such kits may be comprised of nucleic acids encoding a PenetraBody. The nucleic acids may be included in a plasmid or a vector, e.g., a bacterial plasmid or viral vector. Other kits comprise libraries and materials for selecting a PenetraBody.

The present invention provides kits for preventing or treating infectious disease in a patient. For example, a kit may comprise one or more pharmaceutical compositions as described above and optionally instructions for their use. In still other embodiments, the invention provides kits comprising one more pharmaceutical composition and one or more devices for accomplishing administration of such compositions.

Kit components may be packaged for either manual or partially or wholly automated practice of the foregoing methods. In other embodiments involving kits, instructions for their use may be provided.

6. Other Embodiments

In addition to the other embodiments, aspects and objects of the present invention disclosed herein, including the claims appended hereto, the following paragraphs set forth additional, non-limiting embodiments and other aspects of the present invention (with all references to paragraphs contained in this section referring to other paragraphs set forth in this section):

1. A method of making and using PenetraBodies to target internal epitopes in the cytosol for treating chronic infections and diseases in humans wherein said PenetraBodies are composed of a receptor-targeting antibody fragment (R_{AF}), and an antibody fragment that targets an internal epitope available in the cytosol (I_{AF}), wherein the R_{AF} preferentially targets a specific receptor protein on specific cell types, gets internalized by receptor-mediated endocytosis, and then the I_{AF} preferentially targets the intended internal epitope in cytosol.

2. The PenetraBody of paragraph 1, wherein both R_{AF} and I_{AF} domains are structurally stable and functionally active under the reducing conditions of the cytosolic environment.

3. The PenetraBody of paragraph 1, wherein the R_{AF} and I_{AF} domains are independent entities, and are composed of either a single chain antibody (scFv), or a variable domain of light chain (V_{L}), or a variable domain of heavy chain (V_{H}).

4. A method of making PenetraBody of paragraph 1 by fusing the DNA fragment of [R_{AF}], which codes for the R_{AF} domain, with a DNA fragment of [I_{AF}], which codes for the I_{AF} domain, forming thereby a fusion DNA fragment [R_{AF}-I_{AF}], such that it codes for the PenetraBody.

5. A method of making PenetraBody of paragraph 1 by fusing the DNA fragment of [R_{AF}], which codes for the R_{AF} domain, with a DNA fragment [L], which codes for a linker peptide L, and with a DNA fragment [I_{AF}], which codes for the I_{AF} domain, forming thereby a fusion DNA fragment selected from the group consisting of [R_{AF}-L-I_{AF}] and [I_{AF}-L-R_{AF}], such that it codes for the PenetraBody.

6. The method of paragraph 5, wherein the peptide linker is chosen to be short, flexible, hydrophilic, and soluble.

7. The method of paragraph 5, wherein the linker (L) is an endosomal escape domain.

8. A method of making PenetraBody of paragraph 1 by selecting R_{AF} domain that is internalized into target cells through a previously identified internalizing receptor, said method comprising:

a) contacting one or more of said target cells with one or more members of a bacterial display library;

b) contacting members of said bacterial display library with subtractive cell lines;

c) washing said target cells to remove said subtractive cell lines and to remove members of said bacterial display library that are non-specifically bound or weakly bound to said target cells;

d) culturing said target cells under conditions where members of said bacterial display library can be internalized if bound to an internalizing marker; and

e) identifying internalized members of said bacterial display library if members of said bacterial display library are internalized into one or more of said target cells.

9. The method of paragraph 8, wherein said bacterial display library is a HuMAb library wherein the said library displays either single chain variable fragments (scFv), or variable domain of light chains (V_{L}), or variable domain of heavy chains (V_{H}).

10. The method of paragraph 8, wherein said identifying comprises recovering the internalized bacterium and repeating steps (a) through (e) to further select for internalizing R_{AF}, wherein said procedure comprises lysing said target cells to release internalized bacterium and culturing the bacterium for a subsequent round of selection, wherein said procedure comprises recovering the nucleic acid encoding the antibody.

11. The method of paragraph 8, wherein said identifying comprises recovering the internalized bacterium and repeating steps (a) through (e) to further select for internalizing R_{AF}, wherein said procedure comprises sorting target cells with internalized bacterium by FACS.

12. The method of paragraph 8, wherein said identifying comprises detecting expression of a reporter gene or a selectable marker, wherein said cells of a subtractive cell line are present in at least 2-fold excess over said target cells, wherein step (b) is performed at a temperature lower than step (d), wherein step (b) is performed at about 4° C. and step (d) is performed at about 37° C., wherein said bacterium expresses a selectable marker that is selected from the group consisting of a fluorescent protein, an antibiotic resistance gene, and a chromogenic gene, wherein said chromogenic gene is selected from the group consisting of horse radish peroxidase, β-lactamase, luciferase, and β-galactosidase.

13. The method of paragraph 8, wherein said target cells are selected from the group consisting of CD4⁺ T cells, B cells, hepatocytes, members of a cDNA expression library, cells that over express a chemokine receptor, cells of a transformed cell line, and cells transformed with a gene or cDNA encoding a specific surface target receptor.

14. The method of paragraph 8, wherein said subtractive cell lines are selected from the groups consisting of normal human fibroblasts, breast cell lines, and cardiomyocetes.

15. A method of making PenetraBody of paragraph 1, wherein the I_{AF} domain is selected by a method comprising:

(a) contacting one or more of said fluorescently-labeled target antigens with one or more members of a bacterial display library;

(b) expressing the said I_{AF} and its corresponding antigen in bacterial cytoplasm;

(c) selecting high affinity I_{AF} to that particular antigen by flow cytometry; and

(d) selecting the soluble and functional I_{AF} variant in bacterial cytosol.

16. The method of paragraph 15, wherein said bacterial display library is a HuMAb library wherein the said library displays either single chain variable fragments (scFv), or variable domain of light chains (V_{L}), or variable domain of heavy chains (V_{H}).

17. The method of paragraph 15 wherein said method determines whether the I_{AF} component of PenetraBody is soluble and functional under the reducing conditions of bacterial cytosol, which method comprises the steps of:

a) fusing a DNA fragment, [I_{AF}], which codes for the I_{AF} domain of PenetraBody with DNA [R], which codes for a reporter protein, R, and DNA [L], which codes for a linker sequence, L, forming thereby a fusion DNA sequence in either configuration, [I_{AF}-L-R] or [R-L-I_{AF}], which codes for the fusion protein I_{AF}-L-R or R-L-I_{AF}, such that the detection of R signal in I_{AF}-L-R or R-L-I_{AF} indicates that the I_{AF} domain of PenetraBody is soluble;

b) expressing the fusion DNA [I_{AF}-L-R] such that the fusion protein I_{AF}-L-R is produced;

c) detecting reporter protein R in fusion protein I_{AF}-L-R, whereby if reporter protein R is detected in the fusion protein I_{AF}-L-R, the I_{AF} of PenetraBody is soluble; and

d) expressing green fluorescent protein as the reporter protein R, and detecting the fluorescent signal by flow cytometry, and sorting the bacterial cells by fluorescently-activated cell sorting (FACS).

18. The method for paragraph 15 wherein said method modifies the solubility of an I_{AF} domain of PenetraBody in bacterial cytoplasm, which method comprises the steps of:

a) introducing mutations into [I_{AF}], the DNA sequence which codes for said I_{AF} domain, generating thereby a combinatorial library of mutated variants, [X];

b) creating a combinatorial library of mutated variants [X] includes methods selected from the group consisting of recombination, error-prone PCR, propagation in error-prone host strains, site-directed mutation, in vitro scanning saturation mutagenesis, and combinations thereof;

c) in-frame fusing individual [X] antibody variants, with a DNA construct which contains [R] which codes for a reporter protein R which can be detected in solution, forming thereby a set of DNA constructs containing [X-L-R], which code for the fusion proteins, X-L-R, such that the detection of R in an X-L-R protein indicates that the variant antibody, X, contained therein is soluble;

d) expressing each of the DNA constructs such that fusion proteins X-L-R are produced; whereby, if one of the fusion proteins contains a variant X having increased solubility, the reporter protein R exhibits improved detection in X-L-R, thereby indicating that the mutated antibody variant of I_{AF} is more soluble than I_{AF}; and

e) expressing green fluorescent protein as the reporter protein R, and detecting the fluorescent signal by flow cytometry, and sorting the bacterial cells by fluorescently-activated cell sorting (FACS).

19. The pharmaceutical composition comprised of a PenetraBody of paragraph 1, and a pharmaceutically acceptable carrier.

20. A method of inhibiting HCV infection and multiplication in humans, comprising administering to humans an effective amount of the PenetraBody of paragraph 19, wherein the said R_{AF} domain specifically binds a unique receptor, triggers endocytosis, and internalized in hepatocytes and B cells, wherein said receptor is determined by the de novo identification, wherein said receptor is present only in these cells and not present in other cell types, and the said group of receptors includes CD81, SR-B1, low density lipoprotein receptor (LDLr).

21. A method of inhibiting HCV infection and multiplication in humans, comprising administering to humans an effective amount of the PenetraBody of paragraph 19, wherein the said I_{AF} domains specifically bind a highly conserved epitope of one of the following HCV proteins: (a) NS3 protease; (b) NS3 helicase; (c) NS5B RNA dependent RNA polymerase; (d) NS2 zinc-dependent protease; (e) NS4A component of RNA polymerase; (f) NS4B component of RNA polymerase, thereby inhibiting HCV replication, assembly, and multiplication in hepatocytes and B cells.

22. The I_{AF} domain of paragraph 21, wherein the said I_{AF} domain binds to a conformational epitope in the palm subdomain of the HCV RNA-dependent RNA polymerase (NS5B) and that the said I_{AF} domain recognizes native NS5B expressed in the context of the entire HCV polyprotein or subgenomic replicon.

23. A method of inhibiting HIV infection and multiplication in humans, comprising administering to humans an effective amount of the PenetraBody of paragraph 19, wherein the said R_{AF} domain specifically binds a unique receptor, triggers endocytosis, and internalized in CD4⁺ T cells and macrophages, wherein said receptor is determined by the de novo identification, wherein said receptor is present only in these cells and not present in other cell types, and the said group of receptors includes CD4 receptor.

24. A method of inhibiting HIV infection and multiplication in humans, comprising administering to humans an effective amount of the PenetraBody of paragraph 19, wherein the said I_{AF} domains specifically bind a highly conserved epitope of one of the following HIV proteins: (a) reverse transcriptase, (b) protease; (c) Vif protein; (d) ribonuclease H; (e) integrase; (f) regulatory protein, Tat; (g) regulatory protein, Rev; (h) accessory protein, Nef; and (i) the accessory protein, Vpv, thereby inhibiting HIV replication, assembly, and multiplication in CD4⁺ T cells and macrophages.

25. The I_{AF} domain of paragraph 24, wherein the said I_{AF} binds to HIV-1 protease at the epitope, p36-p46, which includes the amino acid sequence LPGRWKPK.

26. The I_{AF} domain of paragraph 24, wherein the said I_{AF} binds to the RNase H domain of the HIV-1 reverse transcriptase and thus arrests the activity of RNA-dependent DNA polymerase activity.

27. The I_{AF} domain of paragraph 24, wherein the said I_{AF} binds to the HIV-1 Gag protein containing the p17-p24 cleavage site and thereby preventing proteolysis of this site by the HIV-1 protease.

28. The epitopes in paragraphs 21 23 are highly conserved conformational or linear epitopes among various clinical isolates of HIV-1 and various genotypes of HCV.

29. The PenetraBody of paragraph 1, wherein the R_{AF} and I_{AF} domains are human monoclonal antibody fragments isolated from a bacterial display library, called non-immune libraries, constructed from healthy individuals free of chronic diseases and infections.

30. The PenetraBody of paragraph 1, wherein the R_{AF} and I_{AF} domains are human monoclonal antibody fragments isolated from a bacterial display library, called immune libraries, constructed from individuals with a chronic disease or infection.

31. The PenetraBody of paragraph 1, wherein the R_{AF} and I_{AF} domains have exceptional serum stability and are functionally active in the said mammalian cytosol at 37° C. for at least 12 hours.

32. The reducing environment of paragraphs 15, 17, and 18 is a bacterial cytoplasm.

33. The bacterial cytoplasm of paragraphs 15, 17, and 18 is that of Escherichia coli.

34. A method of selecting and identifying antibody variants of R_{AF} and I_{AF} domains of paragraph 1 by in vitro scanning saturation mutagenesis, which method comprises the steps of:

a) providing a DNA segment encoding a antibody, or antigen-binding fragment thereof;

b) providing a set of primers that encode all nineteen amino acid variants at a single residue of said antibody or antigen-binding fragment thereof;

c) performing PCR reactions on said DNA segment, using said set of primers, to generate a set of variant DNA segments encoding nineteen amino acid substitution variants at said single residue of said antibody or antigen binding fragments thereof;

d) expressing each of said substitution variants using in vitro transcription/translation;

e) identifying said antibody variant by antigen binding activity.

35. The PenetraBody of paragraph 1, wherein the said R_{AF} and I_{AF} domains are composed of any one of the following:

a) CDR2-CDR3 fusion of the variable fragments of heavy chains (V_{H}),

b) CDR3 of the variable fragments of heavy chains (V_{H}),

c) CDR2-CDR3 fusion of the variable fragments of light chains (V_{L}),

d) CDR3 of the variable fragments of light chains (V_{L}).

### EXAMPLES

The present invention will now be described by way of examples, which are meant to illustrate, but not limit, the scope of the invention.

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publications, for example, Sambrook, Fritsch, and Maniatis, Molecular Cloning: A Laboratory Manual, 2^{nd} Edition, Cold Spring Harbor Laboratory Press, USA, (1989). The practice of the present invention employs, unless otherwise indicated, conventional methods of virology, microbiology, molecular biology, antibody engineering, and recombinant DNA techniques within the skill of the art. Such techniques are explained fully in the literature. See e.g., Current Protocols in Immunology Vol. I-V (Coligan et. al., eds., 2004), John Wiley & Sons, Inc.; Current Protocols in Molecular Biology Vol. I-IV (Ausubel et. al., eds., 2002), John Wiley & Sons, Inc.; Current Protocols in Protein Science Vol. I-III (Coligan et. al., eds., 2001), John Wiley & Sons, Inc.

### Example I

### Generation of HuMAb Bacterial Display Library

Non-immune HuMAb libraries will be constructed by APEx display technology in E. coli. These libraries may be constructed in scFv, V_{H} and V_{L} formats, as necessary. The following commercial sources of RNA will be employed as template: human normal lymph node 5 mg Poly(A)+ RNA pooled from 29 females/males (Clontech, Palo Alto, Calif.); human normal spleen 5 mg Poly(A)+ RNA pooled from 14 females/males (Clontech); human normal spleen 5 mg Poly(A)+ RNA pooled from 7 females/males (Origene, Rockville, Md.); human normal spleen 5 mg Poly(A)+ RNA pooled from 8 females/males (Biochain Inst., Hayward, Calif.). Superscript II (Gibco BRL, Rockville, Md.) first strand synthesis reactions will be set up to generate cDNA from RNA obtained from the fours sources just listed. A total of 16 reactions (4 per each RNA source) will be set up using gene-specific primers to amplify the IgG, IgM heavy chains, and the g and k light chains. The HuIgG, HuIgM, Huk forward and Hul forward primers used for the gene-specific cDNA synthesis are synthesized as described (Marks et. al., J. Mol. Biol. 222:581, 1991). PCR reactions may be set up on the Roche light cycler; the four first-strand synthesis reactions synthesized using the HuIgM primer are pooled, as are the four for HuIgG, three for HuGκ, and four for HuGλ

The primers used for cloning heavy and light chains are published elsewhere (Sblattero and Bradbury, Immunotechnol. 3:271, 1998). Appropriate volumes of the PCR reactions will be mixed together to normalize the amount of material being pooled from each PCR reaction. This will create three pools: λ+κ light chain variable region mix, IgG heavy chain variable region, and IgM heavy chain variable region. The material for the light chain library is digested with EcoRI-BspEI and cloned into a vector, pVL. The library consists of 1.5×10⁷ clones. Approximately 100 colonies will be screened by colony PCR to determine presence of the insert, and will be sequenced. The IgG and IgM heavy chain material will be used to create an IgG and a separate IgM heavy chain library by the parallel processing of the material. The material may be digested with appropriate restriction enzymes, run on an agarose gel, and band-purified before being cloned into a modified pBluescript plasmid (Stratagene, La Jolla, Calif.). Two ligations with 100 ng of cut vector are set up, cleaned up, and eletroporated into electrocompetent E. coli. This will create two libraries, IgM variable region heavy chain (pVM; 3×10⁸ clones) and an IgG variable region heavy chain library (pVG; 6×10⁸ clones). Pfu Polymerase (Stratagene, La Jolla, Calif.) is used for PCR amplification of λκ light chain library, IgG, and IgM.

scFvs will be constructed by overlap extension PCR essentially as described (Sheets, et. al., Proc. Natl. Acad. Sci. USA 95:6157, 1998). Ten 50 μl amplifications will be set up for each one. Approximately 3×10⁸ templates may be used in each reaction (1 ng of supercoiled plasmid containing the heavy chain library or the light chain library). One PCR reaction is set up to join IgG to λκ and another to join IgM to λκ. Pfu-amplified IgG λκ template (50 ng; 2×10¹¹ molecules) will be mixed with 50 ng of Pfu-amplified λκ template. The material will be joined by overlap extension using ten cycles of the following protocol in a 100 μl reaction. A 5 μl sample of the joined heavy and light chain material (˜10¹⁰ molecules) will be added to a 50 μl PCR reaction containing the outside primers of constructs. The materials from both reactions will be pooled in equal amounts and digested with the restriction enzymes NotI and NheI. As applicable, the XhoI site will be replaced with a NotI site because NotI does not cut within any of the V, D, or J genes of either heavy or light chains (Feldhaus et. al., Nature Biotechnol. 21:163, 2003). pAPEx1 will be prepared to accept the insert by double-digesting the vector with appropriate restriction enzymes (Harvey et. al., Proc. Natl. Acad. Sci. USA 101:9193, 2004). All of the resulting cleaned and pooled ligated material may be transformed into ElectroMax-competent E. coli in 25 electroporations. The dilution plates may demonstrate that 2×10⁹ total transformants are generated for the scFv library. Inserts are verified by four-base cutter finger-printing essentially as described (Marks et. al., J. Mol. Biol. 222:581, 1991).

APEX DISPLAY: The leader peptide and first six amino acids of the mature NlpA protein flanked by BdeI and SfiI sites will be amplified by whole cell PCR of XL1-Blue (Stratagene) by using primers as described (Harvey et. al., Proc. Natl. Acad. Sci. USA 101:9193, 2004). The resulting NlpA fragment will be used to replace the pelB leader sequence of pMoPac1 (Hayhurst et. al., J. Immunol. Meth. 276:185, 2003) via NdeI and SfiI to generate pAPEX1. scFvs, and V_{H} and V_{L} domains will be inserted downstream of the NlpA fragment in pAPEx1 via the noncompatible SfiI sites.

E. coli ABLE C (Stratagene) will be the host strain used throughout. E. coli cells transformed with the APEx1 derivatives are inoculated in terrific broth (12 g of pancreatic digest of casein, 9.4 g of dipotassium phosphate, 2.2 g of monopotassium phosphate, pH 7.2) supplemented with 2% glucose and chloramphenicol at 30 μg/ml to an OD₆₀₀ of 0.1. Cell growth and induction will be performed as described (Chen et. al., Nature Biotechnol. 19:537, 2001). After induction, the cellular outer membrane will be permeabilized as described (Hayhurst and Harris, Protein Expr. and Purif. 15:336, 1999). Briefly, cells (equivalent to 1 ml of 20 OD₆₀₀) are pelleted and resuspended in 350 μl of ice-cold solution of 0.75 M sucrose: 0.1 M Tris-Cl, pH 8.0: 100 μg/ml hen egg lysozyme. To this 700 μl of ice-cold 1 mM EDTA will be gently added and the suspension will be left on ice for 10 min; 50 μl of 0.5 M MgCl₂ will be added and the mix may be left on ice for additional 10 min. The resulting cells will be gently pelleted and resuspended in 1×PBS buffer with 200 nM probe at room temperature before evaluation by flow cytometry (FC). Fluorescent probes will be prepared by the procedures as described (Harvey et. al., supra).

Affinity Maturation of scFv Libraries with FC: From parental scFv (and V_{H} or V_{L}), libraries will be made by using error-prone PCR with standard techniques (Fromant et. al., Anal. Biochem. 224:347, 1995) and cloned into the pAPEx1 expression vector. Upon transformation, induction, and labeling, the cells will then stained with propidium iodide (PI; emission 617 nm) to monitor inner membrane integrity. Cells may be analyzed on a MoFlo (Cytomation, Fort Collins, Colo.) droplet deflection flow cytometer by using a 488-nm Argon laser for excitation. Cells will be selected based on improved fluorescence in the fluorescence emission spectrum detecting through 530/40 band-pass filter and for the absence of labeling in PI emission detecting through a 630/40 band-pass filter. E. coli cells captured after the first sort will be immediately resorted through the FC. Subsequently, the scFv genes in the sorted cell suspension will be amplified by PCR. Once amplified, the mutant scFv genes will then be recloned into pAPEx1 vector, retransformed into cells, and grown overnight on agar plates at 30° C. The resulting clones may be subjected to a second round of sorting and re-sorting as described above, before scFv genes are subcloned into pMoPac16 (Hayhurst et. al., J. Immunol. Meth. 276:185, 2003) for expression.

Surface Plasmon Resonance (SPR) Analysis: Monomeric scFv, V_{H}, and V_{L} proteins will be purified by size-exclusion FPLC. Affinity measurements may be obtained via SPR by using a BIACORE 3000 (Bioacore, Uppsala, Sweden) instrument. Approximately 500 response units of the antigen are coupled to a CM5 chip by using 1-ethyl-3-(3-dimethylaminopropyl)carbidiimide/N-hydroxy succinimide chemistry. BSA may be similarly coupled and used for in-line subtraction. Kinetic analysis will be performed at 25° C. in HEPES-buffered saline-EP buffer (Biacore) at a flow rate of 100 μl/min. Five 2-fold dilutions of each antibody beginning at 20 nM will be analyzed in triplicate.

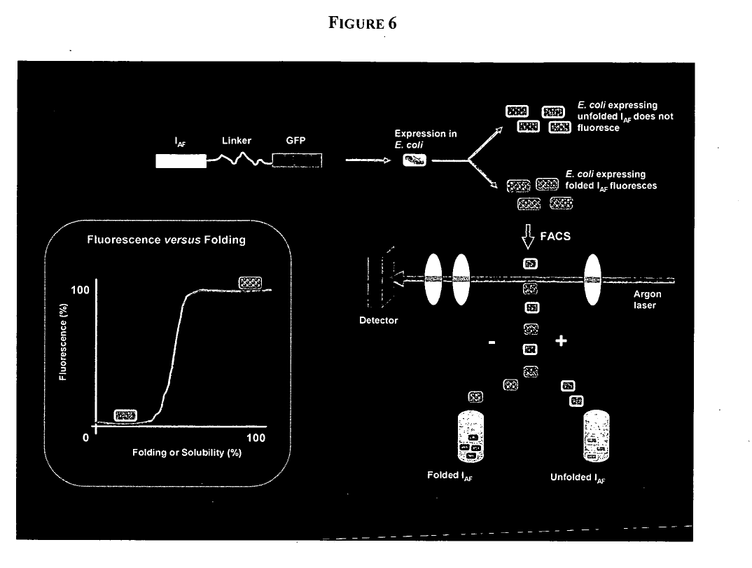

Endogenous production of fluorescent antigens and targeting them to periplasm may be performed by the established protocols (Harvey et. al., Proc. Natl. Acad. Sci. USA 101:9193, 2004). Briefly, GFP-SsrA fusion proteins will be constructed by constructing pTGS30. The plasmid pTGS (DeLisa et. al., J. Biol. Chem. 277:29825, 2002) which contains the arabinose promoter and TorA-GFP-SsrA expression cassette will be digested with BamH1 and HindIII and the fragment cloned into plasmid pBAD30 (Guzman et. al., J. Bacteriol. 177:4121, 1995), which contains the ampicillin resistance gene. In this construct, only mature GFP protein is produced in periplasm by the TAT pathway (FIG. 5). The sequence encoding peptide, CFTFKKEFQNNPNPRSLVK, will be introduced between the TorA leader sequence and N terminus of GFP by overlapping PCR procedures (Harvey et. al. supra). The PCR product may be digested with BamHI and XbaI and then cloned into plasmid pTGS30. In this construct, the peptide-fused GFP protein will be targeted to the periplasm by the TAT pathway. E. coli cells co-expressing a fluorescently labeled antigen and an anchored scFv will be sorted by FC. Overnight cultures of XL1-blue cells may be subcultured into fresh terrific broth (TB) medium at 37° C. and induced with 0.2% arabinose (for protein-GFP production) and 0.2 mM isopropyl β-D-thiogalactoside (for scFv production) in mid-exponential phase of the growth. After 4 h, cells may be collected and permeabilized as above to remove the unbound GFP fused probe in the periplasm. The permeabilized cells may be pelleted by 10 min of centrifugation at 10,000 rpm in an Eppendorf 5415 D centrifuge and then resuspended in 1×PBS buffer. 5 ml of resuspended cells may be diluted into 2 ml of 1×PBS buffer for FACSort analysis.

Where necessary (for example, isolation of I_{AF} domain as set forth in Example III), the scFv (and V_{H}, V_{L} domains) and the GFP-antigen fusions lacking the leader sequence will be expressed in the cytoplasm and whole cell FACSort analysis will be carried out by the established protocols (FIG. 6; Valdivia et. al., Gene 173:47, 1996; Chen et. al., Nature Biotechnol. 19:537, 2001).

### Example II

### Selection of Cell-Specific R_{AF} Domain of PenetraBody

Described here is a method to isolate R_{AF} domains as scFv (or V_{H} or V_{L} formats) that selectively binds a receptor and triggers receptor-mediated endocytosis (FIG. 4). In particular, this example mentions specific cell types representing CD4⁺ T cells and hepatocyte cell lines for the isolation of R_{AF} domains of HIV and HCV PenetraBodies, respectively. The subtractive cell lines that will be used are: normal human fibroblasts and MCF7 cells grown in DMEM, 10% (v/v) fetal bovine serum (FBS) (Hyclone), normal human breast cell line Hs 518Bst (ATCC) in DMEM, 10% fetal calf serum complemented with 10 □g/ml bovine insulin and 30 ng/ml epidermal growth factor (EGF), and B-lymphocyte WI-L2 (ATCC CRL-8062) in RPMI 1640 medium supplemented with 10% FCS. Subtractive cell lines are not infected by HIV and HCV.

The target cell lines used for the HCV infection studies will be: Hep3B (ATCC), Huh 7.5 propagated in Dulbecco modified Eagle medium (DMEM) with 10% fetal bovine serum (McKeating et. al., J. Virol. 78:8496, 2004). Human fetal hepatocytes (HFH) will be grown as described (Lázaro et. al., Hepatology 38:1095, 2003). B cell line isolated from the spleen of an HCV-infected patient may be maintained in standard RPMI medium with 20% FBS without any supplement (Sung et. al., J. Virol. 77:2134, 2003).

The target cell lines used for the HIV infection studies will be: The T-cell line H9 (CFAR; Moulard et. al., Proc. Natl. Acad. Sci. USA 99:6913, 2002), Human SupT1, U38, Jurkat cells grown in RPMI 1640 supplemented with 10% heat-inactivated FBS and penicillin-streptomycin, 293T cells (Schaeffer et. al., J. Virol. 78:1377, 2004), CEMx174 cells and monocyte-derived macrophages (Endres et. al., Science 278:1463, 1997).

Before the first round of panning, the bacterial library will be subtracted with non-specific human cell lines (Gao et. al., J. Immunol. Meth. 274:185, 2003). The scFv-bacterial library, ˜5×10¹² cfu, will be first incubated with ˜1.25×10⁸ subtractive cell lines in 10 ml of culture medium for 1 h at 4° C. After centrifugation, the bacteria-containing supernatant may be incubated with ˜5×10⁷ subtractive cells for 1 h at 4° C. The depleted scFv-bacterial library will be obtained after centrifugation and is ready for panning against the intended target cell lines.

Internalization of scFvs by Bacterial Uptake: Periplasmically-anchored scFvs (V_{H} or V_{L} formats are also tried) in E. coli will be selected for their ability to bind specific receptors in mammalian cells by performing bacterial internalization studies (FIG. 4). Assays for entry of bacteria into mammalian cells (Isberg and Falkow, Nature 317:262, 1985; Isberg et. al., Cell 50:769, 1987), called cellular internalization assays, will be performed as described (Ortega-Barria et. al., Cell 67:411, 1991) with modifications. Briefly, the cultured target cells will be grown at various densities in an antibiotic-free medium ˜20-24 h before use. Permeabilized E. coli cells expressing scFvs on the periplasmic membranes will be prepared as described elsewhere (Harvey et. al., supra). The E. coli cells will be then washed with ice-cold PBS and resuspended in antibiotic-free medium at different concentrations depending on the experimental design. The bacteria will be then incubated with target cells for 2 h at 37° C. in a 5% CO₂ atmosphere for bacterial adhesion and endocytosis. Non-endocytosed bacteria will be lysed by adding appropriate antibiotic (such as kanamycin at 25 μg/ml) to the culture medium for another hour. The target cells will then be washed with PBS buffer and lysed in PBS buffer containing 1% Triton X-100. The supernatant of the lysate, which contained the endocytosed bacteria, will be directly plated on LB agar containing ampicillin at 50 μg/ml concentration. The number of bacterial colonies grown on the plates approximately represents the number of internalized bacteria. Alternatively, GFP protein will be expressed in the E. coli cytoplasm as a marker protein, and when internalized, the mammalian cells fluoresce. The fluorescence intensity as measured by FC of a single mammalian cell is directly proportional to the number of bacteria associated with it (Valdivia et. al., Gene 173:47, 1996). The GFP-fluorescing mammalian cells may be sorted by FACS analysis, and the bacteria amplified after lysing and rescuing.

Paratope Mapping: Paratope is the amino acid framework in scFv (or V_{H} or V_{L}) which is responsible for antigen binding. Identification of an antibody's paratope can be useful for guiding the antibody engineering process. In order to determine the paratope of the R_{AF} and I_{AF} domains of PenetraBodies, a library of point mutants may be created by error-prone PCR of the entire scFv, followed by FACS sorting of non-functional but still well-expressed mutants as per the established procedures (Colby et. al., J. Mol. Biol. 342:901, 2004); the locations of mutations that result in loss of binding are indicative of the paratope location. An APEx display library with an average of one mutation per clone spread over both domains of the scFv (or V_{H}/V_{L} formats) and consisting of approximately 107 clones will be generated by error-prone PCR using Taq polymerase in the presence of MnSO₄. The mutation rate used to generate the paratope mapping library may be low in order to increase the likelihood that the isolated non-functional mutants has only a single point mutation. The presence of multiple point mutations would obscure the contribution of each mutation, and only provides domain-level information about the general location of the paratope when all mutations are constrained to a single domain of the scFv. The library will be labeled with ˜25 times the binding K_{d} in order to brightly label clones with wild-type affinity, resulting in a high level of discrimination between wild-type clones and non-functional mutants. Clones isolated after one round of sorting will be amplified and re-labeled to ensure undetectable binding activity at the excess concentration of antigen used. These clones are sequenced, and each clone is scored into one of three categories: those that have only one mutation; those that have multiple mutations spread over both domains of the scFv; and those that have multiple mutations constrained to a single domain of the scFv. In all clones with only one mutation, the mutation is found to be on the light chain of the scFv, as is the case with the two clones with multiple mutations constrained to a single domain. No non-functional mutants will be isolated that have mutations in V_{L} domain. Isolation of clones with mutations in V_{L} domain is statistically significant for mutations randomly distributed between the domains. The strong bias toward binding-loss mutations occurring only in the light chain is indicative of the light chain contributing the great majority of free energy in the binding interaction.

### Example III

### Selection of Antigen-Specific I_{AF} Domain of PenetraBody

This will be performed in two steps. First, the identification of the I_{AF} that binds to HIV reverse transcriptase (antigen) will be by APEx display coupled with flow cytometry (FIG. 5). The antigen (for example, HIV-1 reverse transcriptase) may be expressed as a GFP-fusion construct and exported to periplasm for FC studies. From these experiments, a high affinity binder (scFv, V_{H} or V_{L}) will be chosen and, as the next step, expressed in cytoplasm. As expected, the scFv will not fold and will not be functional. To address this issue, cytoplasmically-folded and functional scFv (or V_{H}/V_{L} formats) will be evolved through antibody engineering using the procedures set forth in the next section (FIG. 6). For selection experiments, the antigen will be also co-expressed in cytoplasm.

Directed Evolution of Intrabodies in Cytoplasm: The GFP folding reporter may be constructed per established procedure (FIG. 6; Waldo et. al., Nature Biotechnol. 17:691, 1999; U.S. Pat. No. 6,448,087). The BglII/XhoI fragment of pET21 (a+) (Novagen, Madison, Wis.) will be inserted into the corresponding site of pET28(a+), and the BamHI/EcoRI site will be replaced with the DNA fragment GGATCCGCTGGCTCCGCTGCTGGTTCTGGCGAATTC coding for the amino acid linker GSAGSAAGSGEF. Large, bulky, hydrophobic residues will be avoided in designing the linker. A longer linker (GGGS)₄ linker will be also tried but does not appear to change the performance of the folding reporter. A soluble GFP variant will be engineered based on a variant that folds well in E. coli using site-directed mutation to eliminate the internal NdeI and BamHI sites and incorporated the red-shift S65T mutation and the folding mutation F64L (Heim et. al., Nature 373:663, 1995; Cormack et. al., Gene 173:33, 1996;), and inserted into the EcoRI/XhoI site of the vector. The NdeI/BamHI cloning site will be replaced by the frameshift stuffer with three translational stops CATATGTGTTAACTGAGTAGGATCC, the resulting vector digested with NdeI and BamHI to receive inserts.

Fluorescence Measurements and Protein Stability: Cultures will be grown at 37° C. in Luri-Bertani media containing 30 μl/ml kanamycin and induced with 1 mM IPTG. Cells will be diluted to OD₆₀₀=0.15 in 10 mM Tris-Cl and 0.15 M NaCl, pH 7.5 (buffer A), and fluorescence measured using a Perkin-Elmer LS 50B spectrofluorimeter (excitation, 490 nm; emission, 510 nm; bandwidth, 5 nm). Fluorescence will be normalized by dividing by fluorescence of E. coli cells expressing GFP alone. Protein solubility may be determined by SDS-PAGE throughout (Zhang et. al., Prot. Expr. Purif. 12:159, 1998). Briefly, a 3 ml culture of cells will be pelleted in a 1.5 ml Eppendorf tube and washed twice with 1 ml of buffer A. The pellet will be resuspended in 150 μl of buffer A and subjected to two sequences of 10 pulses of sonication, using a Branson Ultrasonics (Danbury, Conn.) model 450 sonicator. The sample will be pelleted by centrifugation between the two pulse sequences. The sonicant may be centrifuged at 14,000 g for 15 min and the supernatant fraction removed by pipeting and saved. The remaining pellet will be washed twice with 1 ml buffer A and finally resuspended in 150 μl buffer A. A 5 μl aliquot of the sample (pellet or supernatant) will be mixed with 5 μl of SDS buffer containing dithiothreitol and heated for 15 min at 100° C. in a waterbath. The denatured proteins will be resolved by SDS-PAGE using a 12.5% acrylamide homogeneous gel (PHAST gel instrument; Amersham Pharmacia Biotech, Piscataway, N.J.), stained by Coomassie brilliant blue dye, and fixed. The gels may be scanned, and densitometry analyzed using NIH Image (http://rsb.info.nih.gov/nih-image/). The total expressed protein will be estimated by summing the integrated density of the soluble and insoluble fractions D_{T}=D_{S}+D_{I}. The soluble fraction is defined as S_{F}=D_{S}/D_{T}, and the insoluble fraction being I_{F}=D_{I}/D_{T}. The SDS sample buffer will be spiked with 2 mg/ml of bovine serum albumin (BSA) to provide an internal density standard compensating for differences in loading volume. Before processing, all integrated sample densities will thus be normalized by the BSA-integrated sample density.

Forward and Backward Evolution: scFv (or V_{H}/V_{L}) genes' PCR amplicons will be DNAse-I digested and in vitro recombined (Zhao and A mold, Nucleic Acid Res. 25:1307, 1997) with the following modifications: Co(II) will be used in place of Mn(II) as the metal cofactor, Pfu(exo−) DNA polymerase will be used for backcrossing for high-fidelity amplification. Reassembled genes will be cloned into the GFP fusion vector, and transformed into DH10B by electroporation, yielding approximately 4×10⁷ unique clones. Plasmids isolated from the plates will be transformed into BL21 (DE3) (Novagen). Cells will be plated directly onto nitrocellulose membranes at a density of about 2,000 transformants/plate, grown at 37° C. for 9-12 h until approximately 1 mm diameter, then the membranes transferred to LB+kanamycin plates containing 1 mM IPTG, and induced for 3 h at 37° C. The 40 brightest clones will be picked, maintained as individual permanents, and as pools. DNA from these optima will be used in subsequent rounds of directed evolution. A total of 10,000 clones will be screened for each cycle of forward evolution (FIG. 6). For backcrossing, amplicons derived by PCR from a plasmid isolation of the pooled optima may be combined in a 1:2 ratio with PCR amplicons of wild-type DNA. DNAse-I digest and subsequent protocols will be as described above for the forward evolution (Waldo et. al., Nature Biotechnol. 17:691, 1999).

### Example IV

### Construction of PenetraBody by Fusing R_{AF} and I_{AF} Domains

Fully functional PenetraBody will be constructed by fusing the genes for R_{AF} and I_{AF} domains in frame linked by a gene sequence encoding for (GGGS)ₙ linker where n=1-4. Large, bulky, hydrophobic residues will be avoided in designing the linker. A longer linker (GGGS)₄ linker will also be tried but may not appear to change the performance of the PenetraBody. The PenetraBody is fully functional with respect to its receptor-binding and internal-epitope binding properties (FIG. 7). Moreover, the serum stability of the PenetraBody will be similar to that of the I_{AF}, and where necessary, the R_{AF} will also be selected by directed evolution techniques in the cytoplasm such that the R_{AF} domain is stable in cytoplasm. Instead of this linker, an endosomal escape domain will be positioned between the R_{AF} and I_{AF} domains by previously established procedures (Fominaya and Wels, J. Biol. Chem. 271:10560, 1996; Uherek et. al., J. Biol. Chem. 273:8835, 1998), and several cytoplasmic localization studies will be conducted to determine the efficiency of such endosomal escape domain in the construction of an active PenetraBody. All PenetraBodies will be expressed in E. coli either in the cytoplasm or periplasm, and purified by established protein purification procedures (Hayhurst and Harris, Prot. Expr. Purif. 15:336, 1999)

### Example V

### Improving the Serum Stability of PenetraBodies

Residues modulating stability will be identified by the following procedure (Arndt et. al., Int. J. Cancer 107:822, 2003). The Kabat numbering system will be used to the published sequence of the V_{H} and V_{L} chains, and respective subgroups (Kabat et. al., NIH Pub. No. 91:3242, 1991) will be determined. “Invariant” residues according to Kabat 23, which occur at frequencies >95% at a given position within the respective subgroups and residues at the V_{H}/V_{L} interface as defined before (Chothia et. al., J. Mol. Biol. 186:651, 1985) will be compared to consensus amino acids. Residues deviating from the consensus will be assigned to their structural role within the “common core” of immunoglobulins (Chothia et. al., J. Mol. Biol. 278:457, 1998) and possible destabilizing effects on the common core studies by homology modeling. The sequences of the wild type scFvs and variants will be subjected to saturated BLAST searches against sequences of known structures of scFv antibodies from the PDB database (www.rcsb.org) using the FFAS-server (http://ffas.ljcrf.edu; Jaroszewski et. al., Protein Sci. 9:1487, 2000). Using coordinates of a reference scFv from the database with highest sequence identity to scFv wild-type, homology models for the wild-type and variants with mutations to respective consensus residues will be constructed with the program MODELLER (Marti-Renom et. al., Ann. Rev. Biophys. Biomol. Struct. 29:291, 2000). Models may be adjusted for steric clashes and subjected to energy minimization. The effect of mutations on the structure will be checked by manual inspection of the models with the software program PY-MOL (www.pymol.org). To determine biophysical stability, aliquots of scFv variants will be incubated at concentrations of 10 μg/ml in 90% human serum for varying periods of time at 37° C. Samples will be frozen at −20° C. at different time points until the end of the experiments. Samples will be subsequently analyzed for binding activity to target cells by flow cytometry.

### Example VI

### De Novo Identification of Internalizing Antibody-Receptor Pairs

A) Whole Cell ELISA: The cell binding activity of scFvs may be detected by cell enzyme-linked immunosorbent assay (ELISA). To wells of a 96-well microtiter plate, will be added ˜5×10⁵ cells. Serially diluted scFvs will be incubated with the cells for 1 h in 100 μl PBS/4% skimmed milk (ELISA buffer) at 4° C. Three washes will be performed by adding 180 μl of ELISA buffer followed by centrifugation. A 1:1000 dilution of anti-Flag mAb M2-horseradish peroxidase (HRP) conjugate will be added and incubated for 30 min at 4° C. After three washes, 100 μl of TMB reagent (Pierce) will be added and developed for 10 min before quenching with an equal volume of 2 M H₂SO₄. The absorbance will be measured at 450 nm with a Thermomax microplate reader (Molecular Devices).

B) Immunoprecipitation and Western Blot: The mammalian cells (293T cells and SupT1 in the case of HIV; Hep3B and human fetal hepatocytes in the case of HCV) will be grown to 90% confluence on 150 mm plates and then lysed by adding 3 ml of lysis buffer (50 mM Tris-Cl (pH 7.4), 150 mM NaCl, 0.5% NP-40 detergent, protease inhibitor cocktail (Sigma), 10 mM iodoacetamide, and 1 mM PMSF) to prepare cell extracts. The cell extracts will then be incubated with 1 ml of agarose beads (Amersham Pharmacia) for 30 min at 4° C. to decrease background binding, loaded onto 1.5 ml of anti-Flag mAb M2-agarose gel conjugate bound to the scFv of interest, and incubated for 4 h at 4° C. with slow rotation. The affinity gel may be washed with wash buffer (same as lysis buffer except the concentration of NP-40 is 0.1%), eluted with 200 mM glycine (pH 2.8), and neutralized with 1 M Tris-Cl (pH 8.0). For Western blots, aliquots of each eluate will be run on SDS-PAGE and after separation proteins will be transferred onto a nitrocellulose membrane. The membranes will be blocked with 5% skimmed milk in PBS and incubated with the primary antibody. The membranes will then be washed with PBS/0.1% Tween-20 and incubated with the secondary antibody conjugated to alkaline phosphatase or HRP and developed with enzyme substrates.

C) Receptor Identification: The immunoprecipitates from the different scFv-cell line (or V_{H}/V_{L} domains instead of scFv) pairings are run on SDS-PAGE and stained by SimpleBlue (Invitrogen). Protein bands are excised from gels, rinsed with water, and dehydrated in methanol for 5 min and then rehydrated with 30% methanol/water for 5 min. After two washes with water for 10 min each, the gel is destained with 100 mM ammonium bicarbonate/30% acetonitrile (pH 8.0) for 10 min until colorless. The gel is then crushed and dried in a centrifugal evaporator. Sequencing grade modified trypsin (Promega) (12.5 ng/μl in 25 mM ammonium bicarbonate, pH 8.0) is added for 1 h at 4° C. and then incubated overnight at 37° C. The resulting peptide mixture is collected and concentrated to a minimal volume in the centrifugal evaporator. An aliquot of the peptide mixture (1 μL) is spotted onto the MALDI target plate and the experiments are conducted by using a PerSeptive Biosystems (Framingham, Mass.) Voyager-STR MALDI-TOF reflectron mass spectrometer. Samples are irradiated with a nitrogen laser (Laser Science) operated at 337 nm and the laser beam is attenuated by a variable attenuator and focused on the sample target. Ions produced in the ion source are accelerated with a deflection voltage of 30 kVolts. The ions are then differentiated according to their m/z using a TOF mass analyzer or the TOF reflectron mass analyzer. Peptide mass maps are searched against theoretically derived maps from proteins in the nonredundant protein database (NCBI) using the ProFound online program (www.proteometrics.com).

### Example VII

### Selective Binding Nature of PenetraBodies

The specificity of the I_{AF} domains of PenetraBodies is determined under in vitro conditions. The I_{AF} domain targeting HIV-1 reverse transcriptase will be tested for its specificity against the following: wild type heterodimeric p66/p51 HIV-1 RT derived from BH-10 clone of HIV-1, RT from HIV-2, murine leukemia virus RT, and porcine endogenous retrovirus RT. These RTs are expressed in bacteria and purified by the procedures as described (Herschhorn et. al., Biochim. Biophys. Acta 1648:154, 2003). RNA-dependent DNA polymerase (RDDP) activity, DNA-dependent DNA polymerase (DDDP) activity, and RNase H activity will be assayed according to established procedures (Hizi et. al., Antimicrob. Agent Chemother. 37:1037, 1993; Hizi and Joklik, J. Biol. Chem. 252:2281, 1977; and Herschhorn et. al., supra). Similarly, the I_{AF} domain targeting the NS5B RNA-dependent RNA polymerase will be tested for its specificity against the following: recombinant HCV from genotypes 1a, 1b, 2-6; classical swine fever virus RdRp; and Poliovirus 3D polymerase according to the published procedures (Moradpour et. al., J. Biol. Chem. 277:593, 2002).

### Example VIII

### Pharmacokinetic and Pharmacodynamic Studies on HCV PenteraBodies in SCID Mouse Model

For the PenetraBody developed as the therapeutic lead targeting NS3 protease, serum half-life, in vivo stability, pharmacokinetic studies, efficacy (antiviral effect), safety and toxicity, dose escalation and dose-related responses, and time-course studies on the reduction of viral load will be investigated in Alb-uPA SCID mouse transplanted with chimeric human livers (Mercer et. al., Nature Medicine 7:927, 2001). In these animals, reconstitution with human hepatocytes was reported to reach ˜50% of the liver cell mass. Inoculation of serum from patients with HCV resulted in persistent HCV viremia in about 75% of mice with high-level human hepatocyte engraftment. HCV RNA could be detected by PCR for up to 35 weeks with titers ranging from 3×10⁴ to 3×10⁶ copies/ml. These viral titers are similar to those found in infected humans. Moreover, an approximately 3-log rise in viral titers after inoculation, detection of viral minus-strand RNA in the liver, and the ability to serially passage the virus through several generations of animals provide convincing evidence for active replication and production of infectious viral progeny in this system.

Alb-uPA SCID chimeric mouse model offers a number of important advantages. HCV infection and replication occur in human hepatocytes, as opposed to animal models such as the chimpanzees, the tamarin, and possibly tupaias. Thus, HCV replication takes place in its authentic environment. Moreover, successful infection could be achieved with viral isolates from different HCV-positive human donors. Therefore, the susceptibility of the system to wild-type virus could be shown. Most importantly, this system allows studying the natural infection process with an easily measurable read out and, therefore, may serve as a robust “in vivo neutralization assay”. Once-a-week dosing regimens are compared for PenetraBodies versus small molecule drugs. Alb-uPA SCID mice will be administered subcutaneously with 2.5-50 mg/kg of PenetraBodies or small molecule drugs on days 0, 7, 14, and 21. Another dosing regimen may involve the administration of drugs twice weekly for 21 days. HCV viral load is monitored relative to a buffer control over 1-3 weeks.

### Example IX

### Pharmacokinetic and Pharmacodynamic Studies on HIV PenetraBodies in SCID Mouse Model

For the HIV PenetraBodies developed as the therapeutic lead, serum half-life, in vivo stability, pharmacokinetic studies, drug dosage cum time-course studies on the reduction of viral load, and the corresponding increase in CD4⁺ T-cell count will be investigated in appropriate animal models. SCID-hu Thy/Liv mouse model uses conjoint implants of human fetal thymus and liver. These implants have been shown to be vascularized and to grow when implanted beneath the kidney capsule, eventually reaching a total mass of 10⁷ to 10⁸ human cells (Namikawa et. al., J. Exp. Med. 172: 1055-1063, 1990). Such growth occurs in 80-90% of recipients and is relatively unaffected by variable parameters in the SCID mouse colony. For periods of time as long as 12 months in vivo, the Thy/Liv organs sustain mutlilineage human hematopoiesis and provide for a continuous source of normal human CD4⁺ T cells. Viral replication can be observed in a time- and dose-dependent manner after inoculation of HIV-1, and in the case of some virus isolates, thymocyte depletion is observed within a 3- to 5-week time interval. Stock preparation of PHA-activated PBMCs, stock preparation of HIV-1 isolates, TCID₅₀ assay for HIV-1, p24 ELISA, construction of SCID-hu Thy/Liv mice, and preparation of drugs for administration to SCID-hu Mice, determination of drug levels in plasma, collection of human graft samples from SCID-hu mice, DNA PCR, FACS analysis for thymocyte depletion, and data analysis will be done according to the published procedures (Rabin et. al., Antimicrob. Agents and Chemother. 40: 755-762, 1996).

Although infection of the Thy/Liv implant can be achieved at low frequency after intravenous or intraperitoneal inoculation of virus, the most reliable means of infecting mice has proved to be direct intrathymic injection of small volumes of standardized viral stocks resulting in the infection of all animals. Dose-dependent levels of HIV-1 (as determined by p24 levels and HIV RNA) and CD4⁺ counts over a period of three to four weeks, as well as circulating plasma levels of the PenetraBodies and small molecule drugs will be evaluated in this mice (Rabin et. al., supra). The concentration of scFvs is determined by ELISA. The data are modeled using WinNonlin software (WinNonLin Pharsight, Mountain View, Calif.) to determine pharmacokinetic parameters using a two-compartment, bolus, first-order elimination model for the intravenous samples. Pharmacokinetic analysis of PenetraBodies are carried out in cynomolgus monkeys according to the established procedures (Chapman et. al., Nature Biotechnol. 17: 780-783, 1999). Blood samples are collected for 3-5 weeks after infusion and the concentration of scFvs is determined by ELISA

Once-a-week dosing regimens are compared for PenetraBodies versus small molecule drugs. SCID Mice will be administered subcutaneously with 2.5-25 mg/kg of PenetraBody or 2.5-25 mg/kg of small molecule drugs on days 0, 7, 14 and 21. Another dosing regimen may involve the administration of drugs twice weekly for 21 days. HIV-1 viral load and CD4⁺ T cell count will be monitored relative to a buffer control over 1-3 weeks.

### REFERENCES

All publications and patents mentioned herein are hereby incorporated by reference in their entireties as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, a long with their full scope of equivalents, and the specification, along with such variations. Such equivalents are intended to be encompassed by the following claims.

What is claimed is:

1. A method of making and using PenetraBodies to target internal epitopes in the cytosol for treating chronic infections and diseases in humans wherein said PenetraBodies are composed of a receptor-targeting antibody fragment (R_{AF}), and an antibody fragment that targets an internal epitope available in the cytosol (I_{AF}), wherein the R_{AF} preferentially targets a specific receptor protein on specific cell types, gets internalized by receptor-mediated endocytosis, and then the I_{AF} preferentially targets the intended internal epitope in cytosol.

2. The PenetraBody of claim 1, wherein both R_{AF} and I_{AF} domains are structurally stable and functionally active under the reducing conditions of the cytosolic environment.

3. The PenetraBody of claim 1, wherein the R_{AF} and I_{AF} domains are independent entities, and are composed of either a single chain antibody (scFv), or a variable domain of light chain (V_{L}), or a variable domain of heavy chain (V_{H}).

4. A method of making PenetraBody of claim 1 by fusing the DNA fragment of [R_{AF}], which codes for the R_{AF} domain, with a DNA fragment of [I_{AF}], which codes for the I_{AF} domain, forming thereby a fusion DNA fragment [R_{AF}-I_{AF}], such that it codes for the PenetraBody.

5. A method of making PenetraBody of claim 1 by fusing the DNA fragment of [R_{AF}], which codes for the R_{AF} domain, with a DNA fragment [L], which codes for a linker peptide L, and with a DNA fragment [I_{AF}], which codes for the I_{AF} domain, forming thereby a fusion DNA fragment selected from the group consisting of [R_{AF}-L-I_{AF}] and [I_{AF}-L-R_{AF}], such that it codes for the PenetraBody.

6. The method of claim 5, wherein the peptide linker is chosen to be short, flexible, hydrophilic, and soluble.

7. The method of claim 5, wherein the linker (L) is an endosomal escape domain.

8. A method of making PenetraBody of claim 1 by selecting R_{AF} domain that is internalized into target cells through a previously identified internalizing receptor, said method comprising:
a) contacting one or more of said target cells with one or more members of a bacterial display library;
b) contacting members of said bacterial display library with subtractive cell lines;
c) washing said target cells to remove said subtractive cell lines and to remove members of said bacterial display library that are non-specifically bound or weakly bound to said target cells;
d) culturing said target cells under conditions where members of said bacterial display library can be internalized if bound to an internalizing marker; and
e) identifying internalized members of said bacterial display library if members of said bacterial display library are internalized into one or more of said target cells.

9. The method of claim 8, wherein said bacterial display library is a HuMAb library wherein the said library displays either single chain variable fragments (scFv), or variable domain of light chains (V_{L}), or variable domain of heavy chains (V_{H}).

10. The method of claim 8, wherein said identifying comprises recovering the internalized bacterium and repeating steps (a) through (e) to further select for internalizing R_{AF}, wherein said procedure comprises lysing said target cells to release internalized bacterium and culturing the bacterium for a subsequent round of selection, wherein said procedure comprises recovering the nucleic acid encoding the antibody.

11. The method of claim 8, wherein said identifying comprises recovering the internalized bacterium and repeating steps (a) through (e) to further select for internalizing R_{AF}, wherein said procedure comprises sorting target cells with internalized bacterium by FACS.

12. The method of claim 8, wherein said identifying comprises detecting expression of a reporter gene or a selectable marker, wherein said cells of a subtractive cell line are present in at least 2-fold excess over said target cells, wherein step (b) is performed at a temperature lower than step (d), wherein step (b) is performed at about 4° C. and step (d) is performed at about 37° C., wherein said bacterium expresses a selectable marker that is selected from the group consisting of a fluorescent protein, an antibiotic resistance gene, and a chromogenic gene, wherein said chromogenic gene is selected from the group consisting of horse radish peroxidase, □-lactamase, luciferase, and □-galactosidase.

13. The method of claim 8, wherein said target cells are selected from the group consisting of CD4⁺ T cells, B cells, hepatocytes, members of a cDNA expression library, cells that over express a chemokine receptor, cells of a transformed cell line, and cells transformed with a gene or cDNA encoding a specific surface target receptor.

14. The method of claim 8, wherein said subtractive cell lines are selected from the groups consisting of normal human fibroblasts, breast cell lines, and cardiomyocetes.

15. A method of making PenetraBody of claim 1 wherein the I_{AF} domain is selected by a method comprising:
(a) contacting one or more of said fluorescently-labeled target antigens with one or more members of a bacterial display library;
(b) expressing the said I_{AF} and its corresponding antigen in bacterial cytoplasm;
(c) selecting high affinity I_{AF} to that particular antigen by flow cytometry; and
(d) selecting the soluble and functional I_{AF} variant in bacterial cytosol.

16. The method of claim 15, wherein said bacterial display library is a HuMAb library wherein the said library displays either single chain variable fragments (scFv), or variable domain of light chains (V_{L}), or variable domain of heavy chains (V_{H}).

17. The method of claim 15 wherein said method determines whether the I_{AF} component of PenetraBody is soluble and functional under the reducing conditions of bacterial cytosol, which method comprises the steps of:
a) fusing a DNA fragment, [I_{AF}], which codes for the I_{AF} domain of PenetraBody with DNA [R], which codes for a reporter protein, R, and DNA [L], which codes for a linker sequence, L, forming thereby a fusion DNA sequence in either configuration, [I_{AF}-L-R] or [R-L-I_{AF}], which codes for the fusion protein I_{AF}-L-R or R-L-I_{AF}, such that the detection of R signal in I_{AF}-L-R or R-L-I_{AF} indicates that the I_{AF} domain of PenetraBody is soluble;
b) expressing the fusion DNA [I_{AF}-L-R] such that the fusion protein I_{AF}-L-R is produced;
c) detecting reporter protein R in fusion protein I_{AF}-L-R, whereby if reporter protein R is detected in the fusion protein I_{AF}-L-R, the I_{AF} of PenetraBody is soluble; and
d) expressing green fluorescent protein as the reporter protein R, and detecting the fluorescent signal by flow cytometry, and sorting the bacterial cells by fluorescently-activated cell sorting (FACS).

18. The method for claim 15 wherein said method modifies the solubility of an I_{AF} domain of PenetraBody in bacterial cytoplasm, which method comprises the steps of:
a) introducing mutations into [I_{AF}], the DNA sequence which codes for said I_{AF} domain, generating thereby a combinatorial library of mutated variants, [X];
b) creating combinatorial library of mutated variants [X] includes methods selected from the group consisting of recombination, error-prone PCR, propagation in error-prone host strains, site-directed mutation, in vitro scanning saturation mutagenesis, and combinations thereof;
c) in-frame fusing individual [X] antibody variants, with a DNA construct which contains [R] which codes for a reporter protein R which can be detected in solution, forming thereby a set of DNA constructs containing [X-L-R], which code for the fusion proteins, X-L-R, such that the detection of R in an X-L-R protein indicates that the variant antibody, X, contained therein is soluble;
d) expressing each of the DNA constructs such that fusion proteins X-L-R are produced; whereby, if one of the fusion proteins contains a variant X having increased solubility, the reporter protein R exhibits improved detection in X-L-R, thereby indicating that the mutated antibody variant of I_{AF} is more soluble than I_{AF}; and
e) expressing green fluorescent protein as the reporter protein R, and detecting the fluorescent signal by flow cytometry, and sorting the bacterial cells by fluorescently-activated cell sorting (FACS).

19. The pharmaceutical composition is comprised of a PenetraBody of claim 1, and a pharmaceutically acceptable carrier.

20. A method of inhibiting HCV infection and multiplication in humans, comprising administering to humans an effective amount of the PenetraBody of claim 19, wherein the said R_{AF} domain specifically binds a unique receptor, triggers endocytosis, and internalized in hepatocytes and B cells, wherein said receptor is determined by the de novo identification, wherein said receptor is present only in these cells and not present in other cell types, and the said group of receptors includes CD81, SR-B1, low density lipoprotein receptor (LDLr).

21. A method of inhibiting HCV infection and multiplication in humans, comprising administering to humans an effective amount of the PenetraBody of claim 19, wherein the said I_{AF} domains specifically bind a highly conserved epitope of one of the following HCV proteins: (a) NS3 protease; (b) NS3 helicase; (c) NS5B RNA dependent RNA polymerase; (d) NS2 zinc-dependent protease; (e) NS4A component of RNA polymerase; (f) NS4B component of RNA polymerase, thereby inhibiting HCV replication, assembly, and multiplication in hepatocytes and B cells.

22. The I_{AF} domain of claim 21, wherein the said I_{AF} domain binds to a conformational epitope in the palm subdomain of the HCV RNA-dependent RNA polymerase (NS5B) and that the said I_{AF} domain recognizes native NS5B expressed in the context of the entire HCV polyprotein or subgenomic replicon.

23. A method of inhibiting HIV infection and multiplication in humans, comprising administering to humans an effective amount of the PenetraBody of claim 19, wherein the said R_{AF} domain specifically binds a unique receptor, triggers endocytosis, and internalized in CD4⁺ T cells and macrophages, wherein said receptor is determined by the de novo identification, wherein said receptor is present only in these cells and not present in other cell types, and the said group of receptors includes CD4 receptor.

24. A method of inhibiting HIV infection and multiplication in humans, comprising administering to humans an effective amount of the PenetraBody of claim 19, wherein the said I_{AF} domains specifically bind a highly conserved epitope of one of the following HIV proteins: (a) reverse transcriptase, (b) protease; (c) Vif protein; (d) ribonuclease H; (e) integrase; (f) regulatory protein, Tat; (g) regulatory protein, Rev; (h) accessory protein, Nef; and (i) the accessory protein, Vpv, thereby inhibiting HIV replication, assembly, and multiplication in CD4⁺ T cells and macrophages.

25. The I_{AF} domain of claim 24, wherein the said I_{AF} binds to HIV-1 protease at the epitope, p36-p46, which includes the amino acid sequence LPGRWKPK (SEQ ID NO: 7).

26. The I_{AF} domain of claim 24, wherein the said I_{AF} binds to the RNase H domain of the HIV-1 reverse transcriptase and thus arrests the activity of RNA-dependent DNA polymerase activity.

27. The I_{AF} domain of claim 24, wherein the said I_{AF} binds to the HIV-1 Gag protein containing the p17-p24 cleavage site and thereby preventing proteolysis of this site by the HIV-1 protease.

28. The epitopes in claims 21 and claim 23 are highly conserved conformational or linear epitopes among various clinical isolates of HIV-1 and various genotypes of HCV.

29. The PenetraBody of claim 1, wherein the R_{AF} and I_{AF} domains are human monoclonal antibody fragments isolated from a bacterial display library, called non-immune libraries, constructed from healthy individuals free of chronic diseases and infections.

30. The PenetraBody of claim 1, wherein the R_{AF} and I_{AF} domains are human monoclonal antibody fragments isolated from a bacterial display library, called immune libraries, constructed from individuals with a chronic disease or infection.

31. The PenetraBody of claim 1, wherein the R_{AF} and I_{AF} domains have exceptional serum stability and are functionally active in the said mammalian cytosol at 37° C. for at least 12 hours.

32. The reducing environment of claims 15, 17, and 18 is a bacterial cytoplasm.

33. The bacterial cytoplasm of claims 15, 17, and 18 is that of Escherichia coli.

34. A method of selecting and identifying antibody variants of R_{AF} and I_{AF} domains of claim 1 by in vitro scanning saturation mutagenesis, which method comprises the steps of:
a) providing a DNA segment encoding a antibody, or antigen-binding fragment thereof;
b) providing a set of primers that encode all nineteen amino acid variants at a single residue of said antibody or antigen-binding fragment thereof;
c) performing PCR reactions on said DNA segment, using said set of primers, to generate a set of variant DNA segments encoding nineteen amino acid substitution variants at said single residue of said antibody or antigen binding fragments thereof;
d) expressing each of said substitution variants using in vitro transcription/translation;
e) identifying said antibody variant by antigen binding activity.

35. The PenetraBody of claim 1, wherein the said R_{AF} and I_{AF} domains are composed of any one of the following:
a) CDR2-CDR3 fusion of the variable fragments of heavy chains (V_{H}),
b) CDR3 of the variable fragments of heavy chains (V_{H}),
c) CDR2-CDR3 fusion of the variable fragments of light chains (V_{L}),
d) CDR3 of the variable fragments of light chains (V_{L}).
